(19) United States
(12) Patent Application Publication
(10) Pub. No.: US 2014/0256939 A1
(43) Pub. Date: Sep. 11, 2014

(54) PROCESS OF PREPARING A QUATERNARY AMMONIUM SALT USING PHOSPHATE

(71) Applicant: Dainippon Sumitomo Pharma Co., Ltd., Osaka (JP)

(72) Inventors: Nobuyuki AE, Osaka-shi (JP); Yuji FUJIWARA, Osaka-shi (JP)

(73) Assignee: Dainippon Sumitomo Pharma Co., Ltd., Osaka (JP)

(21) Appl. No.: 14/201,755

(22) Filed: Mar. 7, 2014

Related U.S. Application Data
(63) Continuation-in-part of application No. 14/041,657, filed on Sep. 30, 2013.
Application No. 14/041,657 is a division of application No. 13/093,985, filed on Apr. 26, 2011, now Pat. No. 8,586,737.
(60) Provisional application No. 61/327,804, filed on Apr. 26, 2010.

(51) Int. Cl. C07D 487/10; C07D 417/12; C07D 417/14

(52) U.S. Cl. CPC C07D 487/10; C07D 417/14; C07D 417/12
USPC 544/231; 544/357; 544/368

(57) ABSTRACT

The present invention relates to a novel process for preparing quaternary ammonium salt derivatives.

### RELATED APPLICATIONS

This application is a continuation-in-part of application Ser. No. 14/041,657, filed Sep. 30, 2013, which is a divisional of application Ser. No. 13/093,985, filed Apr. 26, 2011, now U.S. Pat. No. 8,586,737, which claims the benefit of priority of Provisional Application No. 61/327,804, filed Apr. 26, 2010, all of which are incorporated herein by reference.

### TECHNICAL FIELD

The present invention relates to a process for preparing quaternary ammonium salt derivatives.

### BACKGROUND ART

An imide derivative or a salt thereof whose typical example is a compound of formula (8) mentioned later or an acid addition salt thereof is known to be useful as a medicament for treating schizophrenia, senile psychiatric disorder, bipolar disorder, neurosis, etc. (Patent Reference 1). And, some processes for preparing an imide derivative of the following formula (I):

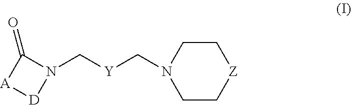

wherein A is optionally substituted C₂₋₄ alkylene group or other, D is carbonyl group or other, Y is optionally substituted C₁₋₂ alkylene group, Z is optionally substituted imino group or other are also reported. For example, Patent Reference 2 discloses a process for preparing the imide derivative of the above-mentioned formula (I) which comprises reacting a compound of formula (II):

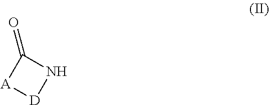

wherein A is optionally substituted C₂₋₄ alkylene group or other, and D is carbonyl group or other, and a quaternary ammonium salt of formula (III):

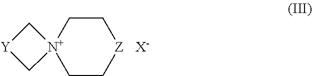

wherein Y is optionally substituted C₁₋₂ alkylene group, Z is optionally substituted imino group or other, X⁻ is a counteranion in the presence of a solid inorganic base and water.

In addition, Patent Reference 3 discloses that the compound of formula (III) can be prepared by reacting a compound of formula (IV):

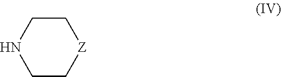

wherein Z is optionally substituted imino group or other, and a compound of formula (V):

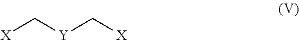

wherein X is a group which can become the above counteranion X⁻ after cleavage, and Y is optionally-substituted C₁₋₂ alkylene group in the presence of potassium carbonate whose specific surface area is less than 1.8 m²/g.

Furthermore, Patent Reference 4 discloses a process for preparing the compound of formula (III) which comprises reacting the compound of formula (IV) and the compound of formula (V) in an organic solvent in the presence of potassium carbonate whose mean particle size (50% D) is not more than 200 μm.

Such compounds and reactions can be used to prepare, for example, lurasidone and salts thereof. FIG. 1 illustrates a synthetic route for the preparation of lurasidone hydrochloride. Referring to the figure, the synthesis can be accomplished in seven steps (labeled P-1 through P-7). Lurasidone, or, lurasidone base, is (1R,2S,3R,4S)—N-[(1R,2R)-2-[4-(1,2-benzisothiazol-3-yl)-1-piperazinylmethyl]-1-cyclohexylmethyl]-2,3-bicyclo[2.2.1]heptanedicarboxyimide. Lurasidone hydrochloride is (1R,2S,3R,4S)—N-[(1R,2R)-2-[4-(1,2-benzisothiazol-3-yl)-1-piperazinylmethyl]-1-cyclohexylmethyl]-2,3-bicyclo-[2.2.1]heptanedicarboxylmide hydrochloride.

In Step P-1, a racemic starting material can be resolved to yield an optically enriched or even optically pure compound. A variety of chiral resolving agents can be used, such as an optically active amine. In some embodiments, for example, optically active ephedrine, optically active norephedrine, optically active pseudoephedrine, optically active N-methylephedrine, optically active 4-hydroxy-norephedrine, optically active 1-phenylethylamine, etc. may be used. In one embodiment, (1S,2R)-(+)-norephedrine may be used. The compounds and methods described in JP 2004-224764 A may also be used.

In Step P-2, the carboxylic groups are esterified. Step P-2 may be carried out using conventional esterification agents, such as an alcohol R⁶—OH and an acidic catalyst. R⁶—OH may be, for example, methanol, ethanol, 1-propanol, 2-propanol, and the like. For an acidic catalyst, sulfuric acid, may be used, for example. The compounds and methods described in JP 2004-224764 A may also be used.

In Step P-3, the ester groups are reduced to alcohols. This step may be carried out by using conventional reducing agents, for example, borohydride compound in an organic solvent, or an aluminum hydride compound in an organic solvent. In some embodiments, sodium borohydride, sodium bis(2-methoxyethoxy)aluminum hydride, lithium aluminum hydride, etc. may be used. Methods described in JP 2005-272335 A or JP 2004-224764 A may also be used.

In Step P-4, the alcohol groups are converted to alkanesulfonates. This step is carried out, for example, by using methanesulfonating agents, such as methanesulfonyl chloride, methanesulfonic anhydride, etc. with an amine, for example, triethylamine, etc. Methods described in JP 2004-224764 A may also be used.

In Steps P-5 and P-6, a quaternary ammonium intermediate and an imide derivative are prepared. These steps may be carried out by using the methods described herein in this specification or US 2011/0263847 A1. Methods described in JP2003-160583A (see, e.g., Examples 1 and 2), JP2006-169154A, JP2006-169155A, JP H8-333368 A or WO 2011/002103 A2 may also be used.

Finally, in Step P-7, a salt of the imide derivative may be prepared using, for example, a pharmaceutically acceptable counterion. This step is carried out by using (i) a mixture of aqueous hydrochloric acid and an organic solvent or (ii) an organic solvent solution of hydrogen chloride. For example, the mixture of aqueous hydrochloric acid and an organic solvent, such as acetone, methyl ethyl ketone, tetrahydrofuran, 2-propanol, etc. may be used. Examples of an organic solvent solution of hydrogen chloride are, for example, hydrogen chloride dissolved in 2-propanol solution and mixed with acetone, hydrogen chloride dissolved in 2-propanol, and the like. Methods described in US 2006/0194970 A1 may also be used.

However, these processes for preparing compounds of formula (I) have some problems on the preparing processes, for example, the product of formula (I) contains a by-product (hereinafter, referred to as “by-product (R)”), or the reaction time of the preparing processes is unstable. Such by-product (R) might cause the quality loss of the imide compound of formula (I), hence it is necessary to remove the by-product through a purification. Thus, it has been desired to further reduce the producing of by-product (R) and stabilize the reaction time from the viewpoint of the yield of the product and the production cost.

### PRIOR ART

### Patent Reference

[Patent Reference 1] JP 2800953 B
[Patent Reference 2] JP 2003-160583 A
[Patent Reference 3] JP 2006-169155 A
[Patent Reference 4] JP 2006-169154 A

### DISCLOSURE OF INVENTION

### Problem to be Solved by the Invention

Under the situation, the present inventors have extensively studied to reduce the producing of by-product (R) and then have found that the cause of producing by-product (R) is potassium carbonate which is used in the reaction of compound (IV) and compound (V) as a base. And, the inventors have further extensively studied other bases instead of potassium carbonate which has been understood as an optimal base in the reaction process and then have found that the producing of by-product (R) can be reduced by using dibasic potassium phosphate with a small amount of water as a base instead of potassium carbonate in the reaction between the following compound of formula (1) and the following compound of formula (2), and the improved process enable the reaction time to be stabilized. Based upon the new findings, the present invention has been completed.

### BRIEF DESCRIPTION OF THE DRAWINGS

FIG. 1 illustrates a reaction synthesis scheme for preparing lurasidone hydrochloride.

FIG. 2A shows the X-ray powder diffraction pattern for 4′-(1,2-benzisothiazol-3-yl)-(3aR,7aR)-octahydro-spiro[2H-isoindole-2,1′-piperazinium]methanesulfonate [Compound (C)], and FIG. 2B shows the peak information for this pattern.

FIGS. 3A-3D show X-ray powder diffraction patterns for lurasidone hydrochloride prepared according to several embodiments disclosed herein, and FIG. 3E shows the peak information for these patterns.

FIGS. 4A-4D show infrared spectra for lurasidone hydrochloride prepared according to several embodiments disclosed herein, and FIG. 4E shows the peak information for these spectra.

FIGS. 5A-5C show differential scanning calorimetry and thermogravimetry graphs for lurasidone hydrochloride prepared according to several embodiments disclosed herein.

FIGS. 6A-6C show X-ray powder diffraction patterns obtained using a synchrotron radiation source for lurasidone hydrochloride prepared according to several embodiments disclosed herein, and FIG. 6D shows the lattice parameter information for these patterns.

FIGS. 7A-7D show X-ray powder diffraction patterns for lurasidone hydrochloride prepared according to several embodiments disclosed herein, and FIG. 7E shows the peak information for these patterns.

FIGS. 8A-8F show X-ray powder diffraction patterns for lurasidone hydrochloride prepared according to an embodiment disclosed herein, compared with the diffraction pattern obtained from a commercially available 40 mg tablet containing lurasidone hydrochloride and with a placebo tablet.

FIGS. 9A-9F show X-ray powder diffraction patterns for lurasidone hydrochloride prepared according to an embodiment disclosed herein, compared with the diffraction pattern obtained from a commercially available 80 mg tablet containing lurasidone hydrochloride and with a placebo tablet.

FIG. 10A shows a X-ray powder diffraction pattern for lurasidone dihydrochloride prepared according to another embodiment disclosed herein, and FIG. 10B shows the peak information for this pattern.

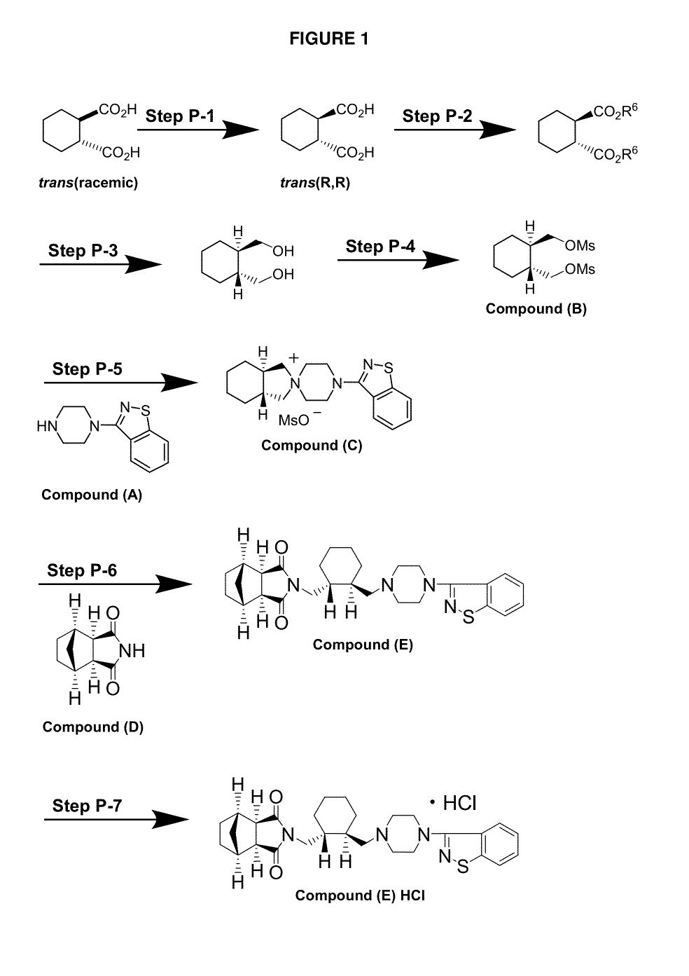

### MEANS TO SOLVE THE PROBLEM

The present inventions are as follows.

### Term 1:

A process for preparing a quaternary ammonium salt of formula (4):

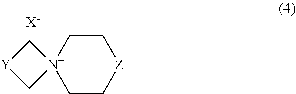

wherein

X is halogen atom, C₁₋₆ alkylsulfonyloxy group, or C₆₋₁₀ arylsulfonyloxy group, and X⁻ is a counteranion thereof,

Y is a substituent of the following formula (3a) or (3b):

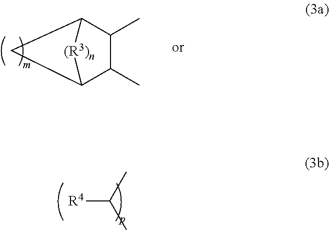

wherein R³ is independently methylene or oxygen atom; R⁴ is independently C₁₋₆ alkyl group, C₁₋₆ alkoxy group, or hydroxy group; m and n are independently 0, 1, 2, or 3; and p is 1 or 2, and

Z is ═N—R¹ or ═CH—R² wherein R¹ is C₁₋₆ alkyl group, C₃₋₇ cycloalkyl group, C₅₋₇ cycloalkenyl group, C₆₋₁₀ aryl group, or 5- to 10-membered monocyclic or bicyclic heteroaryl group; R² is C₁₋₆ alkyl group, C₁₋₆ alkoxy group, C₁₋₆ alkylthio group, C₃₋₇ cycloalkyl group, C₃₋₇ cycloalkyloxy group, C₃₋₇ cycloalkylthio group, C₅₋₇ cycloalkenyl group, C₅₋₇ cycloalkenyloxy group, C₅₋₇ cycloalkenylthio group, C₆₋₁₀ aryl group, C₆₋₁₀ aryloxy group, C₆₋₁₀ arylthio group, 5- to 10-membered monocyclic or bicyclic heteroaryl group, 5- to 10-membered monocyclic or bicyclic heteroaryloxy group, or 5- to 10-membered monocyclic or bicyclic heteroarylthio group,

comprising reacting a compound of formula (1):

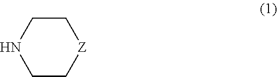

wherein Z is as defined above

with 1 to 2 mole of a compound of formula (2):

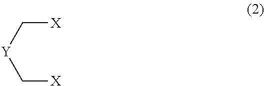

wherein X is independently selected from the above-defined ones, and Y is as defined above, per one mole of the compound of formula (1)

in the presence of 1 to 5 mole of a phosphate per one mole of the compound of formula (1) and 0.01 to 0.1 part by weight of water per one part by weight of the phosphate.

### Term 2:

The process of Term 1 wherein X is independently C₁₋₆ alkylsulfonyloxy group, or C₆₋₁₀ arylsulfonyloxy group.

### Term 3:

The process of Term 2 wherein X is methanesulfonyloxy group.

### Term 4:

The process of any one of Terms 1 to 3 wherein Y is the substituent of formula (3a).

### Term 5:

The process of Term 4 wherein m is 2 and n is 0.

### Term 6:

The process of any one of Terms 1 to 5 wherein Z is ═N—R¹.

### Term 7:

The process of Term 6 wherein R¹ is 5- to 10-membered monocyclic or bicyclic heteroaryl group.

### Term 8:

The process of Term 7 wherein R¹ is 1,2-benzisothiazol-3-yl.

### Term 9:

The process of any one of Terms 1 to 8 wherein the phosphate is dibasic potassium phosphate.

### Term 10:

The process of any one of Terms 1 to 9 wherein the phosphate is 1 to 3 mole per one mole of the compound of formula (1).

### Term 11:

The process of any one of Terms 1 to 10 wherein the amount of water is 0.01 to 0.05 part by weight per one part by weight of the phosphate.

### Term 12:

The process of any one of Terms 1, 9 to 11 wherein the compound of formula (1) is

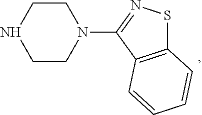 [SMILES: c1ccc2c(N3CCNCC3)nsc2c1]

the compound of formula (2) is

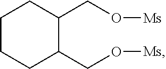 [SMILES: CS(=O)(=O)OCC1CCCCC1COS(C)(=O)=O]

and

the quaternary ammonium salt of formula (4) is

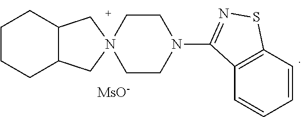

### Term 13:

A process for preparing a compound of formula (8):

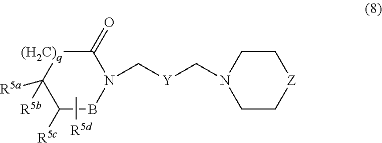

wherein

B is carbonyl group or sulfonyl group,

R^{5a}, R^{5b}, R^{5c}, and R^{5d} are independently hydrogen atom or C₁₋₄ alkyl group, alternatively R^{5a} and R^{5b}, or R^{5a} and R^{5c} may be taken together to form a hydrocarbon ring, or R^{5a} and R^{5c} may be taken together to form an aromatic hydrocarbon ring, wherein the hydrocarbon ring may be bridged with C₁₋₄ alkylene or oxygen atom wherein the C₁₋₄ alkylene and the hydrocarbon ring may be substituted with at least one C₁₋₄ alkyl,

q is 0 or 1, and

Y and Z are as defined in Term 1,

comprising reacting the quaternary ammonium salt (4) prepared according to any one of claims 1 to 12 with the following compound (7):

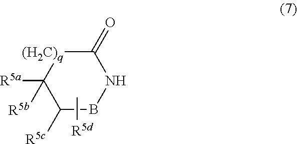

wherein B, R^{5a}, R^{5b}, R^{5c}, R^{5d}, and q are as defined above, in the presence of a solid inorganic base.

### Term 14:

The process of Term 13 wherein B is carbonyl group.

### Term 15:

The process of Term 13 or 14 wherein R^{5a} and R^{5b} are taken together to form a hydrocarbon ring which may be bridged with C₁₋₄ alkylene, and R^{5b} and R^{5d} are hydrogen atom.

### Term 16:

The process of Term 15 wherein Compound (7) is the following compound of formula (7b):

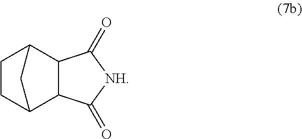 [SMILES: O=C1NC(=O)C2C3CCC(C3)C12]

### Term 17:

The process of any one of Terms 13 to 16 wherein Compound (8) is (3aR,4S,7R,7aS)-2-{(1R,2R)-2-[4-(1,2-benzisothiazol-3-yl)-piperazin-1-ylmethyl]cyclohexylmethyl}hexahydro-4,7-methano-2H-isoindole-1,3-dione.

### Effect of the Invention

According to the present invention, the production of by-product (R) can be held down because the reaction does not include potassium carbonate. In addition, the reaction is carried out with a small amount of water, thereby unfavorable variation of the reaction time caused by such heterogeneous reaction medium can be stabilized. Accordingly, the present reaction can be steadily carried out (i.e. shortening the reaction time and enhancing the transformation rate) and make it possible to prepare quaternary ammonium salt (4) in stably high quality, particularly with an industrial advantage.

### BEST MODE FOR CARRYING OUT THE INVENTION

Hereinafter, the present invention is further illustrated. The number additionally-described in each “substituent” such as “C₁₋₆” means the number of carbons contained therein. For example, “C₁₋₆ alkyl” means an alkyl group having 1 to 6 carbon atoms.

The number of substituents defined in an “optionally substituted” or “substituted” group is not limited as long as the substitution is possible, and the number may be one or more. Each substituent used herein may be applied as a part of other substituent or a substituent of other substituent, unless otherwise indicated.

The term “halogen atom” used herein includes, for example, fluorine atom, chlorine atom, bromine atom and iodine atom, and preferably fluorine atom or chlorine atom.

The term “C₁₋₆ alkyl group” used herein means a straight or branched chain saturated hydrocarbon group having 1-6 carbon atoms, and the preferable one is “C₁₋₄ alkyl group”. The “C₁₋₆ alkyl group” includes, for example, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, and 2-ethylbutyl.

The term “C₃₋₇ cycloalkyl group” used herein means a cyclic saturated hydrocarbon group having 3-7 carbon atoms, and the preferable one is “C₃₋₆ cycloalkyl group”. The “C₃₋₇ cycloalkyl group” includes, for example, cyclopropyl, cyclobutyl, cyclopentyl, and cyclohexyl.

The term “C₆₋₁₀ aryl group” used herein means an aromatic hydrocarbon group having 6-10 carbon atoms, and the preferable one is “C₆ aryl group” (i.e. phenyl). The “C₆₋₁₀ aryl group” includes, for example, phenyl, 1-naphthyl and 2-naphthyl.

The term “C₁₋₆ alkoxy group” used herein means a C₁₋₆ alkyloxy group, wherein the C₁₋₆ alkyl moiety is defined as the above-mentioned “C₁₋₆ alkyl”, and the preferable one is “C₁₋₄ alkoxy group”. The “C₁₋₆ alkoxy group” includes, for example, methoxy, ethoxy, propoxy, isopropoxy, butoxy, isobutoxy, sec-butoxy, and tert-butoxy.

The term “C₃₋₇ cycloalkoxy group” used herein means a C₃₋₇ cycloalkyloxy group, wherein the C₃₋₇ cycloalkyl moiety is defined as the above-mentioned “C₃₋₇ cycloalkyl”. The cyclopropyloxy, cyclobutyloxy, cyclopentyloxy, and cyclohexyloxy.

The “C₆₋₁₀ aryl” moiety in the term “C₆₋₁₀ aryloxy group” used herein is defined as the above-mentioned “C₆₋₁₀ aryl”, and the preferable “C₆₋₁₀ aryloxy group” is “C₆ aryloxy” (i.e. phenyloxy). The “C₆₋₁₀ aryloxy group” includes, for example, phenoxy, 1-naphthyloxy and 2-naphthyloxy.

The “C₁₋₆ alkyl” moiety in the term “C₁₋₆ alkylthio group” used herein is defined as the above-mentioned “C₁₋₆ alkyl”, and the preferable “C₁₋₆ alkylthio group” is “C₁₋₄ alkylthio group”. The “C₁₋₆ alkylthio group” includes, for example, methylthio, and ethylthio.

The “C₃₋₇ cycloalkyl” moiety in the term “C₃₋₇ cycloalkylthio group” used herein is defined as the above-mentioned “C₃₋₆ cycloalkyl”. The “C₃₋₇ cycloalkylthio group” includes, for example, cyclopropylthio, cyclobutylthio, cyclopentylthio, and cyclohexylthio.

The “C₆₋₁₀ aryl” moiety in the term “C₆₋₁₀ arylthio group” used herein is defined as the above-mentioned “C₆₋₁₀ aryl”. The “C₆₋₁₀ arylthio group” includes, for example, phenylthio, 1-naphthylthio and 2-naphthylthio.

The “C₁₋₆ alkyl” moiety in the term “C₁₋₆ alkylsulfonyloxy group” used herein is defined as the above-mentioned “C₁₋₆ alkyl”, and the preferable “C₁₋₆ alkylsulfonyloxy group” is “C₁₋₄ alkylsulfonyloxy group”. The “C₁₋₆ alkylsulfonyloxy group” includes, for example, methylsulfonyloxy, and ethylsulfonyloxy.

The “C₆₋₁₀ aryl” moiety in the term “C₆₋₁₀ arylsulfonyloxy group” used herein is defined as the above-mentioned “C₆₋₁₀ aryl”. The “C₆₋₁₀ arylsulfonyloxy group” includes, for example, phenylsulfonyloxy, 1-naphthylsulfonyloxy and 2-naphthylsulfonyloxy.

The “heteroaryl group” used herein includes, for example, a 5- to 10-membered monocyclic or multi-cyclic aromatic group having one or more heteroatoms (e.g. 1 to 4 heteroatoms) independently-selected from nitrogen, sulfur, and oxygen atom. The “multi-cyclic heteroaryl group” preferably includes a bicyclic or tricyclic one, and more preferably a bicyclic one. The “multi-cyclic heteroaryl group” also includes a fused cyclic group of the above-mentioned monocyclic heteroaryl group with the above-mentioned aromatic ring group (e.g. benzene) or non-aromatic ring group (e.g. cyclohexyl). The “heteroaryl group” includes, for example, the following groups.

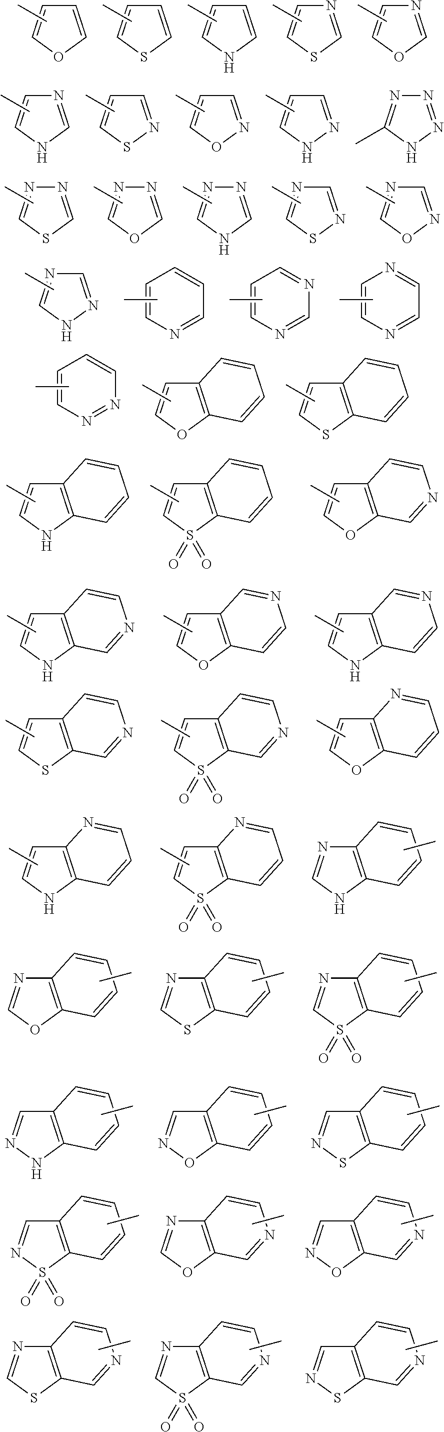

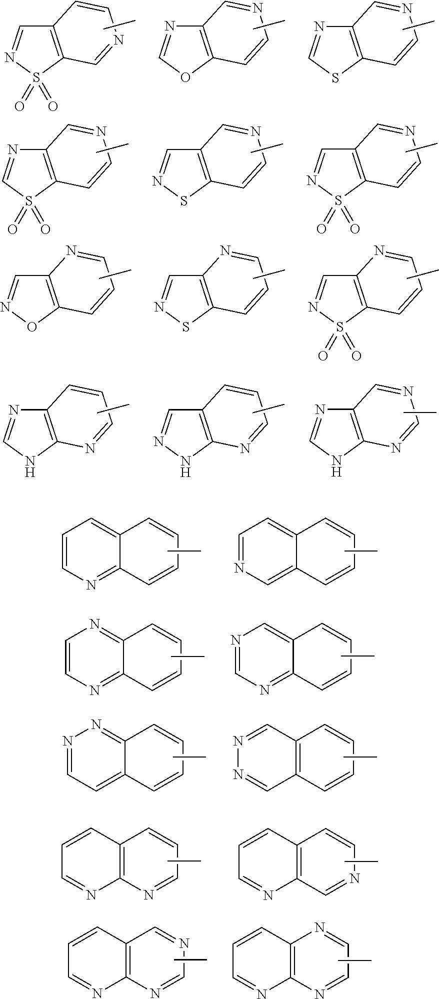

The bond used herein which is connected to the middle of a bond in a ring compound is meant to be attached to any possible position of the ring. For example, the heteroaryl group of the following formula:

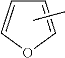

means 2-furyl group, or 3-furyl group.

In case that “heteroaryl group” is a multiple-cyclic group, for example, in case of the following group:

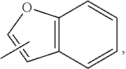

it means 2-benzofuryl group, or 3-benzofuryl group, and additionally, it may mean 4-, 5-, 6- or 7-benzofuryl group. However, in case that a multiple-cyclic heteroaryl group which is composed by fusing an aromatic ring and non-aromatic ring (e.g. piperidine), only the positions in the aromatic ring have the bond. For example, the “multiple-cyclic heteroaryl group” such as the following group:

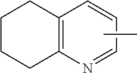

means to be bound on the 2-, 3-, or 4-position.

the “heteroaryl” moiety in the term “heteroaryloxy group” used herein is defined as the above-mentioned “heteroaryl group”. The “heteroaryloxy group” includes, for example, pyridyloxy.

The “heteroaryl” moiety in the term “heteroarylthio group” used herein is defined as the above-mentioned “heteroaryl group”. The “heteroarylthio group” includes, for example, pyridylthio.

The “C₅₋₇ cycloalkenyl group” used herein includes a cycloalkenyl group having 5-7 carbon atoms such as cyclopentenyl group, cyclohexenyl group, and cycloheptenyl group.

The “C₅₋₇ cycloalkenyloxy group” used herein includes a group composed of the above-mentioned cycloalkenyl group and oxygen atom, such as cyclopentenyloxy group.

The “C₅₋₇ cycloalkenylthio group” used herein includes the above-mentioned cycloalkenyloxy group wherein the oxygen atom is replaced by sulfur atom, such as cyclohexylthio group.

The “C₁₋₄ alkylene” used herein has 1-4 carbon atoms and includes, for example, methylene, ethylene, and trimethylene.

The “C₁₋₃ alkylene” used herein has 1-3 carbon atoms and includes, for example, methylene, ethylene, and trimethylene.

The “hydrocarbon ring” used herein is a cyclic alkane having 3-7 carbon atoms such as C₃₋₇ cycloalkane, or a cyclic alkene having 5-7 carbon atoms such as C₅₋₇ cycloalkene. The cyclic alkane having 3-7 carbon atoms includes, for example, cyclopropane, cyclobutane, cyclopentane, cyclohexane, cycloheptane. The cyclic alkene having 5-7 carbon atoms includes, for example, cyclopentene, cyclohexene, and cycloheptene.

The “aromatic hydrocarbon ring” used herein means a ring containing the above-mentioned “C₆₋₁₀ aryl” moiety.

The compound of formula (2) (hereinafter, abbreviated as “Compound (2)”) includes, for example, 1,4-dibromobutane, 1,4-dichlorobutane, 1,4-diiodobutane, 1,4-dimethanesulfonyloxybutane, 1,4-di(p-toluenesulfonyloxy)-butane, 2-hydroxy-1,3-dibromopropane, 2-hydroxy-1,3-dichloropropane, 2-hydroxy-1,3-dimethanesulfonyloxypropane, bis(bromomethyl)cyclohexane, 1,2-bis(methanesulfonyloxymethyl)cyclohexane, 1,2-bis(bromomethyl)cyclopentane, 1,2-bis(methanesulfonyloxymethyl)cyclopentane, 2,3-bis(bromomethyl)-bicyclo[2.2.1]heptane, 2,3-bis(methane-sulfonyloxymethyl)-bicyclo[2.2.1]heptane, 4,5-bis(bromo-methyl)-1-cyclohexene, 4,5-bis(methanesulfonyloxymethyl)-1-cyclohexene, and 2,3-bis(bromomethyl)-7-oxabicyclo[2.2.1]-hept-5-ene.

The “counteranion” includes, for example, halogen ion (e.g. chlorine ion), sulfate ion, hydrogensulfate ion, phosphate ion, hydrogenphosphate ion, dihydrogenphosphate ion, C₁₋₆ alkylsulfonate ion (e.g. methanesulfonate ion), C₁₋₆ arylsulfonate ion (e.g. p-toluenesulfonate ion), and hydroxide ion.

The “by-product which is produced by the reaction with a potassium carbonate of the compound having a carbonate part therein” (by-product (R)) is an all-inclusive term of by-products having at least one carbonate part therein. In the present specification, these by-products are expressed as “by-product (R)”, and the producing rates of by-product (R) in the examples mentioned below are used as an evaluation of the present invention.

In the compound of formula (1) (hereinafter, abbreviated as “Compound (1)”), C₁₋₆ alkyl group, C₃₋₇ cycloalkyl group, C₅₋₇ cycloalkenyl group, C₆₋₁₀ aryl group, and 5- to 10-membered monocyclic or bicyclic heteroaryl group in “R¹”; and C₁₋₆ alkyl group, C₁₋₆ alkoxy group, C₁₋₆ alkylthio group, C₃₋₇ cycloalkyl group, C₃₋₇ cycloalkyloxy group, C₃₋₇ cycloalkylthio group, C₅₋₇ cycloalkenyl group, C₅₋₇ cycloalkenyloxy group, C₅₋₇ cycloalkenylthio group, C₆₋₁₀ aryl group, C₆₋₁₀ aryloxy group, C₆₋₁₀ arylthio group, 5- to 10-membered monocyclic or bicyclic heteroaryl group, 5- to 10-membered monocyclic or bicyclic heteroaryloxy group, and 5- to 10-membered monocyclic or bicyclic heteroarylthio group in “R²” may be further optionally substituted with the same or different one to three substituents selected from the group consisting of C₁₋₄ alkyl, C₁₋₄ alkoxy, C₁₋₄ alkylthio, and halogen atom.

Compound (1) includes, for example, 4-phenylpiperazine, 4-(2-methoxyphenyl)piperazine, 4-cyclohexylpiperazine, 4-(2-pyridinyl)piperazine, 4-(2-pyrimidinyl)piperazine, 4-(2-quinolyl)piperazine, 4-(4-quinolyl)piperazine, 4-(1,2-benzisothiazol-3-yl)piperazine, 4-(4-fluorophenyl)piperidine, 4-[(4-fluorophenyl)thio]-piperidine, 4-(3-chlorophenyl)piperazine, 4-(1,2-benzisoxazol-3-yl)piperidine, 4-(5-benzofuranyl)piperazine, 4-(1-naphthyl)piperazine, 4-[bis(4-fluorophenyl)methylene]-piperidine, 4-(3-isoquinolyl)piperazine, 4-(8-quinolyl)-piperazine, 4-(7-benzofuranyl)piperazine, and 4-(5-fluoro-benzisoxazol-3-yl)piperidine. The preferable example is 4-(1,2-benzisothiazol-3-yl)piperazine.

Compound (1) can be prepared according to, for example, JP 63 (1988)-83085 A, J. Med. Chem., 28761 (1985), and J. Med. Chem., 32, 1024 (1989). And Compound (1) may include an addition acid salt thereof (1) such as a hydrochloride or a sulfate thereof.

As Compound (2) used herein, a commercially available compound may be used. In case that Compound (2) has a chiral carbon(s), i.e. it has an optical isomer, the compound herein may be a single optical isomer, a racemic compound thereof, or a mixture of optical isomers in a certain ratio.

A preferable example of Compound (2) includes a compound of the following formula:

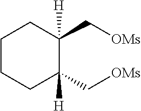

wherein Ms means methanesulfonyl group.

In the reaction between Compound (1) and Compound (2) in the present invention, the amount of Compound (2) used herein is generally 1 mole to 2 mole per one mole of Compound (1). The upper limit amount of Compound (2) used herein is not limited, but, in case that the amount is too much, the process cost increases.

The present invention is directed to the reaction between Compound (1) and Compound (2) using dibasic potassium phosphate with a small amount of water as a base instead of potassium carbonate. The improved process can make the reaction time stabilized and the producing of by-product (R) reduced to prepare a quaternary ammonium salt

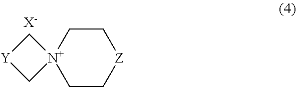

wherein X, Y and Z are as defined in the above Term 1 (hereinafter, abbreviated as “quaternary ammonium salt (4)”) in stably high quality.

The “phosphate” used in the reaction between Compound (1) and Compound (2) includes, for example, an alkali metal phosphate such as potassium phosphate and sodium phosphate; an alkali earth metal salt such as calcium phosphate; and an alkali metal hydrogenphosphate such as dibasic sodium phosphate and dibasic potassium phosphate; preferably dibasic potassium phosphate. Such phosphate may be used alone or as a mixture of two or more kinds of such phosphates. And, such phosphate may be an anhydrous form or a hydrate thereof.

The amount of the phosphate used herein is generally 1.0 mole or more per one mole of Compound (1), and the upper limit amount is not limited, but, in case that the amount is too much, the process cost increases. Accordingly, the amount of the phosphate used is practically 3 mole or less per one mole of Compound (1). And, in case of using an acid addition salt of Compound (1), it is preferable to add an additional appropriate amount of a base to neutralize the acid addition salt. Such base used is generally dibasic potassium phosphate.

The reaction of the present invention is carried out in the coexistence of water, i.e. in the presence of generally 0.01 to 0.1 part by weight, preferably 0.01 to 0.05 part by weight of water per one part by weight of the phosphate. When using a hydrate of dibasic potassium phosphate, the amount of water used herein may be decided considering the water of the hydrate. The water may initially exist in the reaction medium or an appropriate amount of water may be added thereto in mid-course. Or, the water may be added to Compound (1) and/or Compound (2) beforehand.

In addition, the reaction of the present invention may be carried out in the coexistence of a phase-transfer catalyst such as tetra-n-butyl ammonium hydrogen sulfate, tetra-n-butyl ammonium bromide, and benzyl triethyl ammonium chloride. The amount of the phase-transfer catalyst used herein is generally 0.01 to 0.5 mole per one mole of the amount of Compound (1).

In case of using an acid addition salt of Compound (1), it is preferable to add an additional appropriate amount of hydrochloric acid to neutralize the acid addition salt.

The solvent used herein includes, for example, an alcohol solvent such as methanol, and ethanol; an aprotic polar solvent such as acetonitrile, and N,N-dimethylformamide; aromatic carbon ring solvent such as toluene, and xylene; which can be used alone or in a mixture of two or more kinds of the solvents and the amount of the solvent used is not limited.

The reaction temperature is generally 60 to 180° C., preferably 90 to 150° C.

After the reaction is completed, for example, the reaction mixture or a part of the reaction mixture can be concentrated and then filtrated to give a mixture of quaternary ammonium salt (4) and a phosphate. In addition, the reaction mixture containing quaternary ammonium salt (4) and a phosphate may be used in the reaction mentioned below without taking out quaternary ammonium salt (4) from the mixture.

Quaternary ammonium salt (4) thus prepared includes, for example, chloride, bromide, iodide, hydroxide, sulfate, hydrogensulfate, phosphate, hydrogenphosphate, dihydrogen-phosphate, methanesulfonate, and p-toluenesulfonate of
7-cyclohexyl-2-hydroxy-7-aza-4-azoniaspiro[3.5]-nonane,
8-phenyl-8-aza-5-azoniaspiro[4.5]decane,
8-(2-methoxyphenyl)-8-aza-5-azoniaspiro[4.5]decane,
8-(2-pyridinyl)-8-aza-5-azoniaspiro[4.5]decane,
8-(2-pyrimidinyl)-8-aza-5-azoniaspiro[4.5]decane,
8-(2-quinolyl)-8-aza-5-azoniaspiro[4.5]decane,
8-(4-quinolyl)-8-aza-5-azoniaspiro[4.5]decane,
8-(1,2-benzisothiazol-3-yl)-8-aza-5-azoniaspiro-[4.5]decane,
4′-(1,2-benzisothiazol-3-yl)octahydro-spiro[2H-isoindole-2,1′-piperazinium],
4′-[(4-fluorophenyl)thio]octahydro-spiro[2H-isoindole-2,1′-piperidinium],
4′-(2-pyrimidinyl)octahydro-spiro[2H-isoindole-2,1′-piperazinium],
4′-(4-fluorophenoxy)octahydro-spiro[2H-isoindole-2,1′-piperidinium],
4′-(1,2-benzisoxazol-3-yl)octahydro-spiro[2H-isoindole-2,1′-piperidinium],
4′-(6-fluoro-1,2-benzisoxazol-3-yl)-octahydro-spiro[2H-isoindole-2,1′-piperazinium],
4′-(2-pyridinyl)octahydro-spiro[2H-isoindole-2,1′-piperazinium],
4′-(3-chlorophenyl)octahydro-spiro[2H-isoindole-2,1′-piperazinium],
4′-(5-benzofuranyl)octahydro-spiro[2H-isoindole-2,1′-piperazinium],
4′-(1-naphthyl)octahydro-spiro[2H-isoindole-2,1′-piperazinium],
4′-[bis(4-fluorophenyl)methylene]octahydro-spiro[2H-isoindole-2,1′-piperidinium],
4′-(2-methoxyphenyl)octahydro-spiro[2H-isoindole-2,1′-piperazinium],
4′-(3-isoquinolyl)octahydro-spiro[2H-isoindole-2,1′-piperazinium],
4′-(8-quinolyl)octahydro-spiro[2H-isoindole-2,1′-piperazinium],
4′-(1,2-benzisothiazol-3-yl)tetrahydro-spiro-[cyclopenta[c]pyrrole-2(1H),1′-piperazinium],
4′-(1,2-benzisothiazol-3-yl)octahydro-spiro[4,7-methano-2H-isoindole-2,1′-piperazinium],
4′-(1,2-benzisothiazol-3-yl)-1,3,3a,4,7,7a-hexahydro-spiro[2H-isoindole-2,1′-piperazinium],
4′-(1,2-benzisothiazol-3-yl)-1,3,3a,4,7,7a-hexahydro-spiro[4,7-epoxy-2H-isoindole-2,1′-piperazinium], or
4′-(7-benzofuranyl)octahydro-spiro[2H-isoindole-2,1′-piperazinium].

By reacting the resulting quaternary ammonium salt and a compound of formula (7):

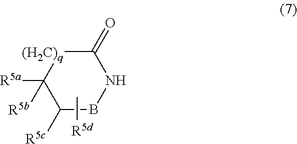

wherein the symbols are as defined in the above Term 13 (hereinafter, abbreviated as “Compound (7)”) in the presence of a solid inorganic base, an imide compound of formula (8):

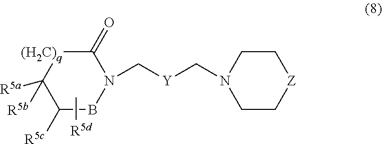

wherein the symbols are as defined in the above Term 13 (hereinafter, abbreviated as “imide compound (8)”) can be prepared.

Compound (7) includes a compound of the following formula (7a):

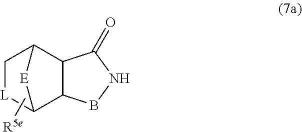

wherein -L- is a single or double bond, E is C₁₋₃ alkylene optionally substituted with C₁₋₄ alkyl or oxygen atom, R^{5e} is hydrogen atom or C₁₋₄ alkyl group, and B is as defined in the above formula (7).

Compound (7) includes, for example, succinimide, 2,6-piperidine-dione, 4,4-dimethyl-2,6-piperidine-dione, 8-azaspiro[4.5]decane-7,9-dione, perhydroazepin-2,7-dione, maleimide, phthalimide, tetrahydrophthalimide, cis-1,2-cyclohexane-dicarboximide, trans-1,2-cyclohexane-dicarboximide, cis-1,2-cyclohex-4-ene-dicarboximide, trans-1,2-cyclohex-4-ene-dicarboximide, cis-4-methyl-1,2-cyclohexane-dicarboximide, trans-4-methyl-1,2-cyclohexane-dicarboximide, cis-1,2-dimethyl-1,2-cyclohexane-dicarboximide, trans-1,2-dimethyl-1,2-cyclohexane-dicarboximide, cis-4,5-dimethyl-1,2-cyclohexane-dicarboximide, trans-4,5-dimethyl-1,2-cyclohexane-dicarboximide, cis-3,6-dimethyl-1,2-cyclohexane-dicarboximide, trans-3,6-dimethyl-1,2-cyclohexane-dicarboximide, bicyclo[2.2.1]heptane-2,3-di-exo-carboximide, bicyclo[2.2.1]heptane-2,3-di-endo-carboximide, bicyclo[2.2.1]hept-5-ene-2,3-di-exo-carboximide, bicyclo[2.2.1]hept-5-ene-2,3-di-endo-carboximide, bicyclo-[2.2.2]octane-2,3-di-exo-carboximide, bicyclo[2.2.2]octane-di-endo-carboximide, bicyclo[2.2.2]oct-5-ene-2,3-di-exo-carboximide, bicyclo[2.2.2]oct-5-ene-2,3-di-endo-carboximide, bicyclo[2.2.2]oct-7-ene-2,3-di-exo-carboximide, bicyclo[2.2.2]oct-7-ene-2,3-di-endo-carboximide, hexahydro-4,7-methano-1,2-benzisothiazol-3(2H)-one-1,1-dioxide,3,6-epoxy-1,2-cyclohexane-dicarboximide, and spiro[bicyclo[2.2.2]octane-2,3′-pyrrolidine]-2′,5′-dione.

A preferable example of Compound (7) includes a compound of the following 7(b):

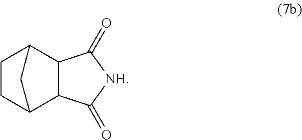 [SMILES: O=C1NC(=O)C2C3CCC(C3)C12]

Compound (7b) can include its optical isomers, thus the compound used herein may be one of the optical isomers or a mixture of the optical isomers. A preferable example of Compound (7) includes a compound of the following formula:

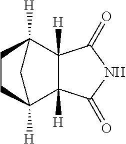 [SMILES: O=C1NC(=O)[C@H]2[C@@H]3CC[C@@H](C3)[C@@H]12]

or a salt thereof.

Compound (7) can be prepared, for example, by reacting a corresponding carboxylic anhydride compound and ammonia (for example, JP-1(1989)-199967 A).

The solid inorganic base (salt) includes, for example, an alkali metal carbonate such as potassium carbonate, and sodium carbonate; an alkali earth metal salt such as calcium carbonate, and magnesium carbonate; and an alkali metal bicarbonate such as sodium bicarbonate, and potassium bicarbonate; preferably an alkali metal carbonate, in particular, potassium carbonate. Such solid inorganic base may be used alone or as a mixture of two or more kinds of bases. And, such solid inorganic bases may be an anhydrous form or a hydrate thereof.

The amount of the solid inorganic base used herein is generally 0.7 mole or more, preferably 0.9 mole or more per one mole of the amount of Compound (1) or quaternary ammonium salt (4). The upper limit amount of the solid inorganic base used herein is not limited, but, in case that the amount is too much, the process cost increases. Accordingly, the practical amount of the solid inorganic base is 3 mole or less, preferably 2.7 mole or less per one mole of the amount of Compound (1) or quaternary ammonium salt (4).

The amount of Compound (7) used herein is generally 0.7 mole or more per one mole of the amount of Compound (1) or quaternary ammonium salt (4). The upper limit amount of Compound (7) used herein is not limited, but, in case that the amount is too much, the process cost increases. Accordingly, the practical amount of Compound (7) is 2.5 mole or less per one mole of the amount of Compound (1) or quaternary ammonium salt (4).

The reaction of the present invention is generally carried out in the presence of a solvent. The solvent used herein includes, for example, aromatic hydrocarbons such as toluene, xylene, mesitylene, chlorobenzene, and dichlorobenzene. The amount of such solvent used herein is generally 3 parts by weight or more, preferably 5 parts by weight or more per one part by weight of the total amount of Compound (1) or quaternary ammonium salt (4). The upper limit amount of the solvent used herein is not limited, but, in case that the amount is too much, the volumetric efficiency is turned down. Accordingly, the practical amount of the solvent is 20 parts by weight or less per one part by weight of the amount of Compound (1) or quaternary ammonium salt (4).

The reaction of the present invention is preferably carried out in the coexistence of water, i.e. in the presence of generally 0.05 to 3 mole, preferably 0.1 to 1.5 mole of water per one mole of the amount of Compound (1) or quaternary ammonium salt (4). When using a hydrate of solid inorganic base, the amount of water used herein may be decided considering the water of the hydrate. The water may initially exist in the reaction medium or an appropriate amount of water may be added thereto in mid-course. Or, the water may be added to Compound (7) and/or quaternary ammonium salt (4) beforehand.

In addition, the reaction of the present invention may be carried out in the coexistence of a phase-transfer catalyst such as tetra-n-butyl ammonium hydrogen sulfate, tetra-n-butyl ammonium bromide, and benzyl triethyl ammonium chloride. The amount of the phase-transfer catalyst used herein is generally 0.01 to 0.5 mole per one mole of the amount of Compound (2) or quaternary ammonium salt (4).

The reaction temperature is generally 80 to 180° C., preferably 95 to 150° C.

The reaction of quaternary ammonium salt (4) and Compound (7) is generally carried out by contacting and mixing quaternary ammonium salt (4), Compound (7) and a solid inorganic base, and the addition order of the substances is not limited. The solid inorganic base may be added thereto in separated amounts or in a lump, but it is preferable in a lump.

The reaction mixture containing imide compound (8) is obtained after the reaction, and the mixture can be treated by adding water thereto, mixing it, standing still in a whole, separating it with a separating funnel, optionally treating the organic layer with active carbon, and concentrating the organic layer to give imide compound (8). Alternatively, imide compound (8) can be obtained as a crystal by cooling the above-mentioned organic layer or the partially-concentrated organic layer, or adding another solvent which is comparatively insoluble for imide compound to the organic layer. The solvent which is comparatively insoluble for imide compound (8) includes, for example, an aliphatic hydrocarbon solvent such as pentane, hexane, and heptane, and an alcohol solvent such as methanol, ethanol, and isopropanol.

In addition, imide compound (8) can be also obtained from the reaction mixture containing imide compound (8) by removing out insoluble precipitates with a filter and concentrating the filtrate. Further, imide compound (8) can be obtained as a crystal by cooling the reaction mixture or the partially-concentrated reaction mixture, or adding another solvent which is comparatively insoluble for imide compound (8) to the organic layer.

The obtained imide compound (8) may be further purified by a conventional purification such as recrystallization and chromatography. In addition, imide compound (8) can be obtained as an inorganic acid addition salt such as hydrochloride, sulfate, hydrobromide, and phosphate; or an organic acid addition salt such as acetate, oxalate, citrate, malate, tartrate, maleate, and fumarate.

The imide compound (8) prepared herein includes, for example,
2-[4-(4-phenyl-1-piperazinyl)butyl]hexahydro-1H-isoindole-1,3(2H)-dione,
2-[4-(4-phenyl-1-piperazinyl)butyl]hexahydro-4,7-methano-1H-isoindole-1,3(2H)-dione,
2-[4-[4-(2-methoxyphenyl)-1-piperazinyl]butyl]hexahydro-1H-isoindole-1,3(2H)-dione,
2-[4-[4-(2-methoxyphenyl)-1-piperazinyl]butyl]hexahydro-4,7-methano-1H-isoindole-1,3(2H)-dione,
2-[4-[2-[[4-(1,2-benzisothiazol-3-yl)-1-piperazinyl]-methyl]cyclohexyl]methyl]hexahydro-4,7-methano-1H-isoindole-1,3(2H)-dione (2-[2-[4-(1,2-benzisothiazol-3-yl)-piperazin-1-ylmethyl]cyclohexylmethyl]hexahydro-4,7-methano-2H-isoindole-1,3-dione),
2-[[2-[[4-(1,2-benzisothiazol-3-yl)-1-piperazinyl]-methyl]cyclohexyl]methyl]hexahydro-4,7-methano-1,2-benzisothiazole-3(2H)-one-1,1-dioxide,
2-[[2-[[4-(1,2-benzisothiazol-3-yl)-1-piperazinyl]-methyl]cyclohexyl]methyl]hexahydro-1H-isoindole-1,3(2H)-dione,
2-[[2-[[4-(2-pyrimidinyl)-1-piperazinyl]methyl]-cyclohexyl]methyl]hexahydro-4,7-methano-1H-isoindole-1,3(2H)-dione,
2-[[2-[[4-(1,2-benzisothiazol-3-yl)-1-piperazinyl]methyl]cyclohexyl]methyl]-3a,4,7,7a-tetrahydro-1H-isoindole-1,3(2H)-dione,
8-[[2-[[4-(1,2-benzisothiazol-3-yl)-1-piperazinyl]methyl]cyclohexyl]methyl]-8-azaspiro[4,5]decane-7,9-dione,
1-[[2-[[4-(1,2-benzisothiazol-3-yl)-1-piperazinyl]methyl]cyclohexyl]methyl]-4,4-dimethyl-2,6-piperidine-dione,
2-[[2-[[4-(1,2-benzisothiazol-3-yl)-1-piperazinyl]methyl]cyclohexyl]methyl]hexahydro-4,7-epoxy-1H-isoindole-1,3(2H)-dione,
1′-[[2-[[4-(1,2-benzisothiazol-3-yl)-1-piperazinyl]methyl]cyclohexyl]methyl]-spiro[bicyclo[2.2.2]octane-2,3′-pyrrolidine]-2′,5′-dione,
2-[[2-[[4-(1,2-benzisothiazol-3-yl)-1-piperazinyl]methyl]cyclohexyl]methyl]hexahydro-3a,7a-dimethyl-1H-isoindole-1,3(2H)-dione,
2-[[2-[[4-(1,2-benzisothiazol-3-yl)-1-piperazinyl]methyl]cyclohexyl]methyl]-3a,4,7,7a-tetrahydro-4,7-ethano-1H-isoindole-1,3(2H)-dione,
2-[[2-[[4-(1,2-benzisothiazol-3-yl)-1-piperazinyl]methyl]cyclohexyl]methyl]hexahydro-4,7-ethano-1H-isoindole-1,3(2H)-dione,
2-[[2-[[4-(1,2-benzisothiazol-3-yl)-1-piperazinyl]methyl]cyclohexyl]methyl]-1H-isoindole-1,3(2H)-dione,
2-[[2-[[4-(1,2-benzisothiazol-3-yl)-1-piperazinyl]methyl]cyclohexyl]methyl]-4,5,6,7-tetrahydro-1H-isoindole-1,3(2H)-dione,
2-[[2-[[4-[(4-fluorophenyl)thio]-1-piperidyl]methyl]cyclohexyl]methyl]hexahydro-4,7-methano-1H-isoindole-1,3(2H)-dione,
2-[[2-[[4-[(4-fluorophenyl)thio]-1-piperidyl]methyl]cyclohexyl]methyl]hexahydro-1H-isoindole-1,3(2H)-dione,
2-[[2-[[4-(4-fluorophenoxy)-1-piperidyl]methyl]cyclohexyl]methyl]hexahydro-4,7-methano-1H-isoindole-1,3(2H)-dione,
2-[[2-[[4-(4-fluorophenoxy)-1-piperidyl]methyl]cyclohexyl]methyl]hexahydro-1H-isoindole-1,3(2H)-dione,
2-[[2-[[4-(1,2-benzisoxazol-3-yl)-1-piperidyl]methyl]cyclohexyl]methyl]hexahydro-4,7-methano-1H-isoindole-1,3(2H)-dione,
2-[[2-[[4-(1,2-benzisoxazol-3-yl)-1-piperidyl]methyl]cyclohexyl]methyl]hexahydro-1H-isoindole-1,3(2H)-dione,
2-[[2-[[4-(6-fluoro-1,2-benzisoxazol-3-yl)-1-piperidyl]methyl]cyclohexyl]methyl]hexahydro-4,7-methano-1H-isoindole-1,3(2H)-dione,
2-[[2-[[4-(6-fluoro-1,2-benzisoxazol-3-yl)-1-piperidyl]methyl]cyclohexyl]methyl]hexahydro-1H-isoindole-1,3(2H)-dione,
2-[[2-[[4-(2-pyridinyl)-1-piperazinyl]methyl]cyclohexyl]methyl]hexahydro-4,7-methano-1H-isoindole-1,3(2H)-dione,
2-[[2-[[4-(2-pyridinyl)-1-piperazinyl]methyl]cyclohexyl]methyl]hexahydro-1 H-isoindole-1,3(2H)-dione,
2-[[2-[[4-(2-pyrimidinyl)-1-piperazinyl]methyl]cyclohexyl]methyl]hexahydro-4,7-methano-1H-isoindole-1,3(2H)-dione,
2-[[2-[[4-(2-pyrimidinyl)-1-piperazinyl]methyl]cyclohexyl]methyl]hexahydro-1H-isoindole-1,3(2H)-dione,
2-[[2-[[4-(3-chlorophenyl)-1-piperazinyl]methyl]cyclohexyl]methyl]hexahydro-4,7-methano-1H-isoindole-1,3(2H)-dione,
2-[[2-[[4-(3-chlorophenyl)-1-piperazinyl]methyl]cyclohexyl]methyl]hexahydro-1H-isoindole-1,3(2H)-dione,
2-[[2-[[4-(5-benzofuranyl)-1-piperazinyl]methyl]cyclohexyl]methyl]hexahydro-4,7-methano-1H-isoindole-1,3(2H)-dione,
2-[[2-[[4-(5-benzofuranyl)-1-piperazinyl]methyl]cyclohexyl]methyl]hexahydro-1H-isoindole-1,3(2H)-dione,
2-[[2-[[4-(1-naphthyl)-1-piperazinyl]methyl]cyclohexyl]methyl]hexahydro-4,7-methano-1H-isoindole-1,3(2H)-dione,
2-[[2-[[4-(1-naphthyl)-1-piperazinyl]methyl]cyclohexyl]methyl]hexahydro-1 H-isoindole-1,3(2H)-dione,
2-[[2-[[4-[bis(4-fluorophenyl)methylene]-1-piperidyl]methyl]cyclohexyl]methyl]hexahydro-4,7-methano-1H-isoindole-1,3(2H)-dione,
2-[[2-[[4-[bis(4-fluorophenyl)methylene]-1-piperidyl]methyl]cyclohexyl]methyl]hexahydro-1H-isoindole-1,3(2H)-dione,
2-[[2-[[4-(2-methoxyphenyl)-1-piperazinyl]methyl]cyclohexyl]methyl]hexahydro-4,7-methano-1H-isoindole-1,3(2H)-dione,
2-[[2-[[4-(2-methoxyphenyl)-1-piperazinyl]methyl]cyclohexyl]methyl]hexahydro-1H-isoindole-1,3(2H)-dione,
2-[[2-[[4-(3-isoquinolyl)-1-piperazinyl]methyl]cyclohexyl]methyl]hexahydro-4,7-methano-1H-isoindole-1,3(2H)-dione,
2-[[2-[[4-(3-isoquinolyl)-1-piperazinyl]methyl]cyclohexyl]methyl]hexahydro-1 H-isoindole-1,3(2H)-dione,
2-[[2-[[4-(8-quinolyl)-1-piperazinyl]methyl]cyclohexyl]methyl]hexahydro-4,7-methano-1H-isoindole-1,3(2H)-dione,
2-[[2-[[4-(8-quinolyl)-1-piperazinyl]methyl]cyclohexyl]methyl]hexahydro-1H-isoindole-1,3(2H)-dione,
2-[[2-[[4-(1,2-benzisothiazol-3-yl)-1-piperazinyl]methyl]cyclopentyl]methyl]hexahydro-4,7-methano-1H-isoindole-1,3(2H)-dione,
2-[[2-[[4-(1,2-benzisothiazol-3-yl)-1-piperazinyl]methyl]cyclopentyl]methyl]hexahydro-1H-isoindole-1,3(2H)-dione,
2-[[3-[[4-(1,2-benzisothiazol-3-yl)-1-piperazinyl]-methyl]bicyclo[2.2.1]hept-2-yl]methyl]hexahydro-4,7-methano-1H-isoindole-1,3(2H)-dione,
2-[[3-[[4-(1,2-benzisothiazol-3-yl)-1-piperazinyl]methyl]bicyclo[2.2.1]hept-2-yl]methyl]hexahydro-1H-isoindole-1,3(2H)-dione,
2-[[2-[[4-(7-benzofuranyl)-1-piperazinyl]methyl]cyclohexyl]methyl]hexahydro-4,7-methano-1H-isoindole-1,3(2H)-dione,
2-[[2-[[4-(7-benzofuranyl)-1-piperazinyl]methyl]cyclohexyl]methyl]hexahydro-1H-isoindole-1,3(2H)-dione,
2-[[3-[[4-(1,2-benzisothiazol-3-yl)-1-piperazinyl]methyl]-7-oxabicyclo[2.2.1]hept-5-ene-2-yl]methyl]-hexahydro-4,7-methano-1H-isoindole-1,3(2H)-dione,
2-[[3-[[4-(1,2-benzisothiazol-3-yl)-1-piperazinyl]methyl]-7-oxabicyclo[2.2.1]hept-5-ene-2-yl]methyl]-hexahydro-1H-isoindole-1,3(2H)-dione,
2-[[6-[[4-(1,2-benzisothiazol-3-yl)-1-piperazinyl]methyl]-3-cyclohexen-1-yl]methyl]hexahydro-4,7-methano-1H-isoindole-1,3(2H)-dione, and
2-[[6-[[4-(1,2-benzisothiazol-3-yl)-1-piperazinyl]methyl]-3-cyclohexen-1-yl]methyl]hexahydro-1H-isoindole-1,3(2H)-dione.

In case that the optically active compound (7) and/or the optically active quaternary ammonium salt (4) are used in the reaction, the optically active corresponding imide compound (8) can be obtained.

In addition, the present invention includes the following process:

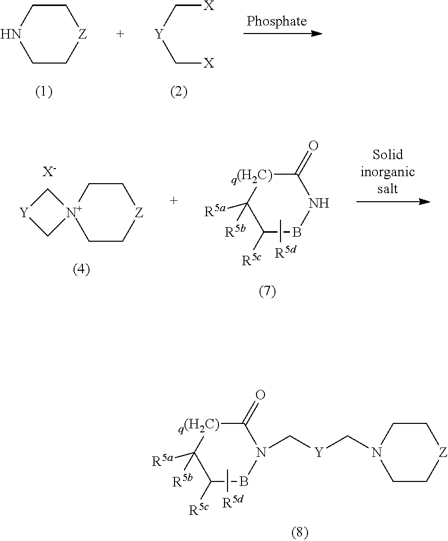

wherein the symbols described in the scheme are as defined in Terms 1 and 13 mentioned above.

### EXAMPLES

Hereinafter, the present invention is illustrated in more detail by the following Examples and Comparative Examples, but it should not be construed to be limited thereto. The analyses in the examples were done by high-performance liquid chromatography (LC).

### Example 1

To a mixed solution of 4-(1,2-benzisothiazol-3-yl)piperazine [Compound (A)] (20.0 g, 91.2 mmol), (1R,2R)-1,2-bis(methanesulfonyloxymethyl)cyclohexane [Compound (B)] (32.9 g, 109.5 mmol), and toluene (280 g) were added dibasic potassium phosphate (47.7 g, 273.9 mmol), water (1.4 g, 77.8 mmol) and tetra-n-butyl ammonium hydrogen sulfate (1.2 g, 3.5 mmol). The mixture was stirred under reflux for 15 hours (water (0.5 g) was added in mid-course) to give a reaction mixture containing 4′-(1,2-benzisothiazol-3-yl)-(3aR,7aR)-octahydro-spiro[2H-isoindole-2,1′-piperazinium]methanesulfonate [Compound (C)].

### Example 2

To the reaction mixture containing Compound (C) which was obtained in the above Example 1 were added (3aR,4S,7R,7aS)-hexahydro-4,7-methano-2H-isoindole-1,3-dione [Compound (D)] (22.6 g, 136.8 mmol), potassium carbonate (15.1 g, 109.3 mmol) and toluene (44 g). Then, the toluene (44 g) was distilled out from the mixture, water (0.82 g) was added thereto, and the resulting mixture was reacted under reflux for 8 hours. Then, the reaction mixture was cooled to room temperature, and water (400 g) was added to the mixture. The mixture was separated with a separating funnel, and the toluene layer was washed with 2.3% (W/W) brine (350 g). Further, active carbon (1.8 g) was added to the toluene solution, and the mixture was stirred for 1 hour. The active carbon was removed by filtration to give a toluene solution containing (3aR,4S,7R,7aS)-2-{(1R,2R)-2-[4-(1,2-benzisothiazol-3-yl) piperazin-1-ylmethyl]cyclohexylmethyl}hexahydro-4,7-methano-2H-isoindole-1,3-dione (2-[[(1R,2R)-2-[[4-(1,2-benzisothiazol-3-yl)-1-piperazinyl]methyl]cyclohexyl]-methyl]hexahydro-(3aS,4R,7S,7aR)-4,7-methano-1H-isoindole-1,3(2H)-dione) [Compound (E)] (341.4 g). The yield of Compound E was 94.3%. The yield of Compound (E) was calculated based on the analytical result that the content of the compound in the toluene solution was 12.4% (w/w) (which was calculated by LC absolute calibration curve method). And, the production rate of by-product (R) was 0.013% (which was calculated with the following formula (a)).

$$Production rate of by \text{-} product derived from carbonate = \frac{Total LC area of by \text{-} product derived from carbonate}{Total LC area of detected peaks except solvent} \times 100 \qquad ( a )$$

### Example 3

To a mixture of Compound (A) (20.0 g, 91.2 mmol), Compound (B) (32.9 g, 109.5 mmol) and toluene (280 g) was added dibasic potassium phosphate (23.8 g, 136.6 mmol), water (0.95 g, 52.8 mmol) and tetra-n-butyl ammonium hydrogen sulfate (1.2 g, 3.5 mmol). The mixture was stirred under reflux for 14 hours to give a reaction mixture containing Compound (C).

### Example 4

To the reaction mixture containing Compound (C) which was obtained in the above Example 3 were added Compound (D) (22.6 g, 136.8 mmol) and potassium carbonate (15.1 g, 109.3 mmol), and the mixture was stirred under reflux for 6 hours. Then, the reaction mixture was cooled to room temperature, and water (400 g) was added to the mixture. The mixture was separated with a separating funnel, and the toluene layer was washed with 2.3% (W/W) brine (350 g). Further, active carbon (1.8 g) was added to the toluene solution, and the mixture was stirred for 1.5 hours. The active carbon was removed by filtration to give a toluene solution containing Compound (E) (415.4 g). The yield of Compound E was 88.6%. The yield of Compound (E) was calculated based on the analytical result that the content of the compound in the toluene solution was 9.6% (w/w) (which was calculated by LC absolute calibration curve method). And, the production rate of by-product (R) was 0.019% (which was calculated with the above formula (a)).

### Example 5

Compound (C) was isolated as a crystalline solid according to the following procedure. A mixture of 4-(1,2-benzisothiazol-3-yl)piperazine [Compound (A)] (120 g, 547 mmol), (1R,2R)-1,2-bis(methanesulfonyloxymethyl)cyclohexane [Compound (B)] (197.2 g, 656 mmol), potassium carbonate (45.4 g, 328 mmol), and toluene (1680 g) was refluxed for about 8 hr. Potassium carbonate (22.7 g, 164 mmol) and tetra-n-butylammonium hydrogen sulfate (7.43 g, 21.9 mmol) were added, and the mixture was further refluxed for about 10 hr. The reaction mixture was cooled and the precipitate was filtered, washed with toluene (2×200 mL), and dried under reduced pressure at 40° C. to give crude 4′-(1,2-benzisothiazol-3-yl)-(3aR,7aR)-octahydro-spiro[2H-isoindole-2,1′-piperazinium]methanesulfonate [Compound (C)] (317.48 g).

A second reaction was performed at one half the scale. A crude product of compound (C) (161.25 g) was obtained from 60 g of Compound (A) using the same protocol.

Crude Compound (C) from the above two preparations were combined (317.48 g+161.25 g), extracted three times with hot acetonitrile (3 L each), and the extracts were mixed and partly concentrated. The resulting precipitate was filtered at room temperature, washed with acetonitrile (2×50 mL) and dried under reduced pressure at 40° C. to give Compound (C) (305.02 g) as a white crystalline solid.

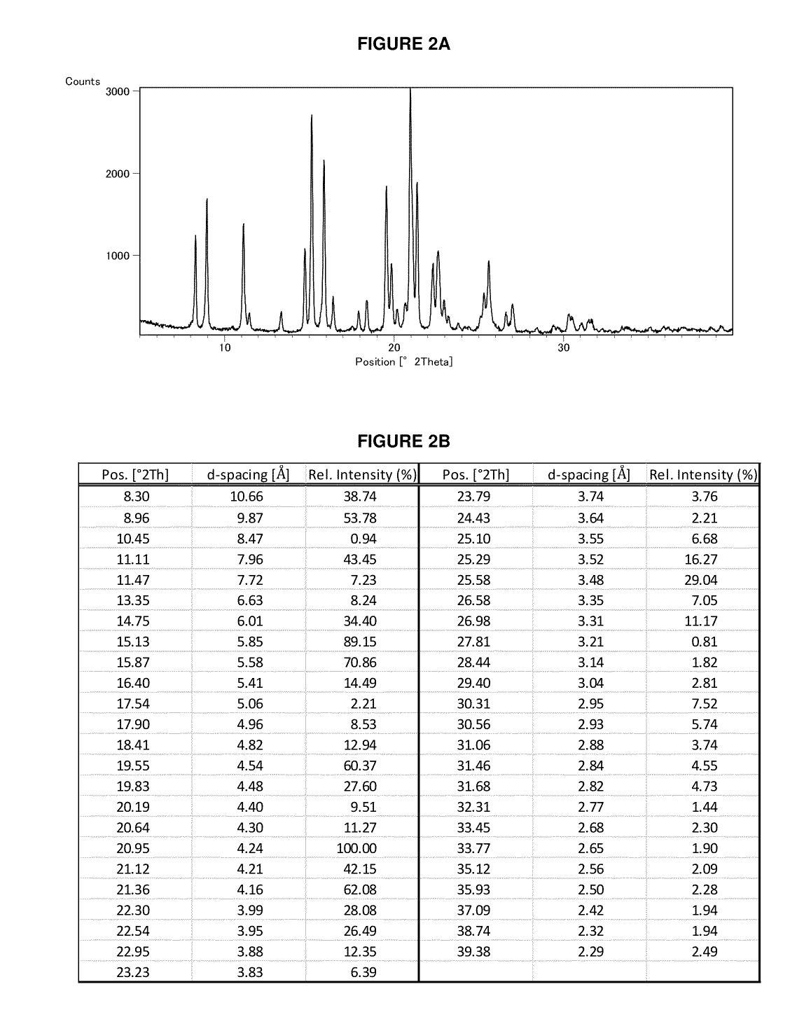

The XRPD pattern for Compound (C) of Example 5 is shown in FIG. 2A and the peak information)(2θ(°), d-spacing, relative intensity) is provided in FIG. 2B.

### Example 6

Compound (E), lurasidone, is obtained in free form as a solid by the following procedure. The toluene solution of Compound (E) as prepared by Example 4 above is concentrated until the weight of the concentrate is 2.23 times the theoretical yield expected for lurasidone. To the concentrate, methanol (5.46 times the weight of the theoretical yield of lurasidone) and seed crystals of lurasidone are added at 55±5° C. After adding additional methanol (3.64 times the weight of the theoretical yield of lurasidone), the slurry is cooled to 3±2° C., filtered, and washed with methanol. The crystals are dried at a maximum temperature of 50° C. Lurasidone can similarly be isolated from the toluene solution thereof described in US 2011/0263847, Examples 2, 4, 6, and 8, and can also be prepared according to the methods described in JP 2003-160583 A, Examples 1 and 2.

### Comparative Example 1

To a mixture of Compound (A) (140.1 kg, 638.8 mol), Compound (B) (230.3 kg, 766.7 mol) and toluene (2272 kg) was added potassium carbonate (53.0 kg, 383.5 mol), the toluene (312 kg) was removed by heating, and then the mixture was reflux-dehydrated for 5 hours. Then, the reaction mixture was cooled to 70° C. or lower, and potassium carbonate (26.5 kg, 191.7 mol) and tetra-n-butyl ammonium hydrogen sulfate (8.7 kg, 25.6 mol) were added to the mixture. The mixture was refluxed for 10 hours to give the reaction mixture containing Compound (C).

### Comparative Example 2

To the reaction mixture containing Compound (C) which was obtained in the above Comparative Example 1 were added toluene (309.6 kg), Compound (D) (158.3 kg, 958.3 mol) and potassium carbonate (105.9 kg, 766.2 mol), and then the toluene (308 kg) was removed by heating. Then, the reaction mixture was cooled to 70° C. or lower, and water (5.7 kg) was added to the mixture. The mixture was refluxed for 4 hours. The reaction mixture was cooled, and water (2819 kg) was added to the mixture. The mixture was separated with a separating funnel, and the toluene layer was washed with 2.3% (w/w) brine (2466 kg). Further, active carbon (12.5 kg) was added to the toluene solution, and the mixture was stirred for 1 hour. The active carbon was removed by filtration and washed with toluene to give a toluene solution containing Compound (E)(2562 kg). The yield of Compound (E) was 87.7%. The yield of Compound (E) was calculated based on the analytical result that the content of the compound in the toluene solution was 10.8% (w/w) (which was calculated by LC absolute calibration curve method). And, the production rate of by-product (R) was 9.83 (which was calculated with the above formula (a)).

### Comparative Example 3

To a mixture of Compound (A) (90.0 kg, 410.4 mol), Compound (B) (147.9 kg, 492.4 mol) and toluene (1460 kg) were added potassium carbonate (34.0 kg, 246.0 mol) and water (636 g), the toluene (298 kg) was removed by heating, and then the mixture was reflux-dehydrated for 34 hours. Then, the reaction mixture was cooled to 70° C. or lower, and potassium carbonate (17.0 kg, 123.0 mol) and tetra-n-butyl ammonium hydrogen sulfate (5.6 kg. 16.5 mol) were added to the mixture. The mixture was refluxed for 12 hours to give the reaction mixture containing Compound (C). And, the production rate of by-product (R) was 3.02% (which was calculated with the above formula (a)).

### Comparative Example 4

To the reaction mixture containing Compound (C) which was obtained in the above Comparative Example 3 were added toluene (198 kg), Compound (D) (101.7 kg, 615.7 mol) and potassium carbonate (68.1 kg, 492.7 mol), and then the toluene (198 kg) was removed by heating. Then, the reaction mixture was cooled to 70° C. or lower, and water (3.7 kg) was added to the mixture. The mixture was refluxed for 3 hours. The reaction mixture was cooled, and water (1803 kg) was added to the mixture. The mixture was separated with a separating funnel, and the toluene layer was washed with 2.3% (w/w) brine (1578 kg). Further, active carbon (8.0 kg) was added to the toluene solution, and the mixture was stirred for 1 hour. The active carbon was removed by filtration and washed with toluene to give a toluene solution containing Compound (E) (1625 kg). The yield of Compound (E) was 90.1%. The yield of Compound (E) was calculated based on the analytical result that the content of the compound in the toluene solution was 11.2% (w/w) (which was calculated by LC absolute calibration curve method). And, the production rate of by-product (R) was 3.08% (which was calculated with the above formula (a)).

Each reaction time, product yield, and by-product yield in the above examples and comparative examples is shown in the following table.

| | | | dibasic | | | | |
|---|---|---|---|---|---|---|---|
| | | | potassium | Potassium | | | |
| | | Compound (B) | phosphate | carbonate | Reaction time | Product yield | By-product (R) |
| | Process | (mol) | (mol) | (mol) | (hr) | (%) | (%) |
| Example 1 | (A) | 1.2 | 3.0 | — | 15 | | |
| Example 2 | (B) | | — | 1.2 | 8 | 94 | 0.013 |
| Example 3 | (A) | 1.2 | 1.5 | — | 14 | | |
| Example 4 | (B) | | — | 1.2 | 6 | 89 | 0.019 |
| Comparative | (A) | 1.2 | — | 0.9 | 15 | | |
| Example 1 | | | | | | | |
| Comparative | (B) | | — | 1.2 | 4 | 88 | 9.83 |
| Example 2 | | | | | | | |
| Comparative | (A) | 1.2 | — | 0.9 | 46 | | 3.02 |
| Example 3 | | | | | | | |
| Comparative | (B) | | — | 1.2 | 3 | 90 | 3.08 |
| Example 4 | | | | | | | |

Process (A): Compound (A) + Compound (B) --> quaternary ammonium salt (C)
Process (B): quaternary ammonium salt (C) + Compound (D) --> imide compound (E)

According to the results of Examples 1 and 3, the process of the present invention can make the reaction time for preparing quaternary ammonium salt (4) shortened, i.e. the reaction times in all the examples could be steadily shortened in 15 hours. In addition, the production of by-product (R) could be drastically held down by the present invention. Accordingly, the process of the present invention is an industrially useful manufacturing method which is also for practical preparation.

The practical application of compounds prepared herein is further illustrated by the following examples, which describe the preparation and characterization of solid forms of lurasidone hydrochloride, as well as comparisons of the x-ray diffraction patterns of these compounds with those of commercial samples.

Analyses of the purity of lurasidone HCl in the following examples were performed by HPLC using a YMC-Pack Pro C18 column (5 μm, 6.0 mm Φ×15 cm), with two mobile phases, with Pump A: 5 mM phosphate buffer (pH 7)/acetonitrile (4:1), and Pump B: 5 mM phosphate buffer (pH 7) operated under the following run conditions:

| Time (min) | Pump A (%) | Pump B (%) |
|---|---|---|
| 0.0 | 50.0 | 50.0 |
| 5.0 | 50.0 | 50.0 |
| 35.0 | 13.0 | 87.0 |
| 50.0 | 13.0 | 87.0 |

X-ray powder diffraction (XRPD) experiments were conducted on samples powdered in an agate mortar and then mounted on a Si plate. For X-ray diffraction measurements made on a tablet, the tablet was affixed to the sample holder using MYLAR® polyester film. XRPD data were collected using a Bruker D8 Advance (Billerica, Mass.) with a Cu source (1.54 Å) at a voltage of 40 kV and a current of 40 mA.

Additional XRPD measurements were conducted at SPring-8 (Super Photon ring, 8 GeV synchrotron radiation source) in Harima Science Park. Samples were powdered in an agate mortar and packed into a Lindeman glass capillary (0.3-0.7 mm diameter). The capillary was mounted on the Debye-Schere camera (BL19B2), radius of 286.5 mm, with an imaging plate as a detector. Diffraction patterns were recorded in the 2θ range of 0-75°. The synchrotron source wavelength was 1.299 Å, and the exposure time was 5-15 min for one diffraction image. The data was analyzed and lattice parameters calculated using PDXL, version 2.1.3.4 (Rigaku).

To prepare lurasidone tablets (40 mg or 80 mg), the methods for preparing film-coated tablets described in US 2009/0143404 A1 were used. Generally, granules of lurasidone HCl, mannitol, pregelatinized starch, croscarmellose sodium, hypromellose and magnesium stearate were prepared, and a tableting machine was used to compress the granules into tablets. The tablets were film-coated by using coating pans from above the tablets and film-coating agents such as hypromellose, titanium oxide, polyethylene glycol and carnauba wax. Placebo tablets were prepared in a similar manner, except that lurasidone HCl was replaced with mannitol. In some cases, the 80 mg tablets were prepared with yellow ferric oxide and FD&C Blue No. 2 Aluminum Lake.

Infrared spectra (IR) were obtained from solid samples prepared by triturating the sample compound (1-2 mg) with potassium chloride (100 mg) while minimizing moisture absorption, and compressing the mixture in a press to form a sample disk. The IR spectra were obtained using IRPrestige-21 (Shimadzu Corp., Kyoto, Japan) at 2 cm⁻¹ resolution.

Differential scanning calorimetry (DSC) analyses were performed using a DSC-Q-1000 (TA Instruments, New Castle, Del.). The scanning rate was 10 K/min and the sample weight 2-5 mg. Samples were packed in aluminum hermetic pans.

Thermogravimetric analyses (TGA) were performed using a DSC-Q-500 (TA instruments, New Castle, Del.). The scanning rate was 10 K/min and the sample weight was 10-20 mg. Samples were mounted on a platinum pan.

### Example 7

Lurasidone (8.25 g) was dissolved in acetone (102 g) with heating under reflux to give an acetone solution thereof. This solution was added dropwise to a 3.6% aqueous hydrochloric acid solution (18.7 g, 1.1 equivalents) over a period of one hour while the solution was kept at about 55° C. After the addition was completed, the reaction mixture was stirred at about 60° C. for one hour. The reaction mixture was cooled to 0° C., and stirred at the same temperature for one hour. The mixture was filtered, and the resulting solid was dried at room temperature under reduced pressure to give lurasidone hydrochloride (7.67 g, yield: 86.6%). HPLC analysis of the product demonstrated the purity was 99.97%.

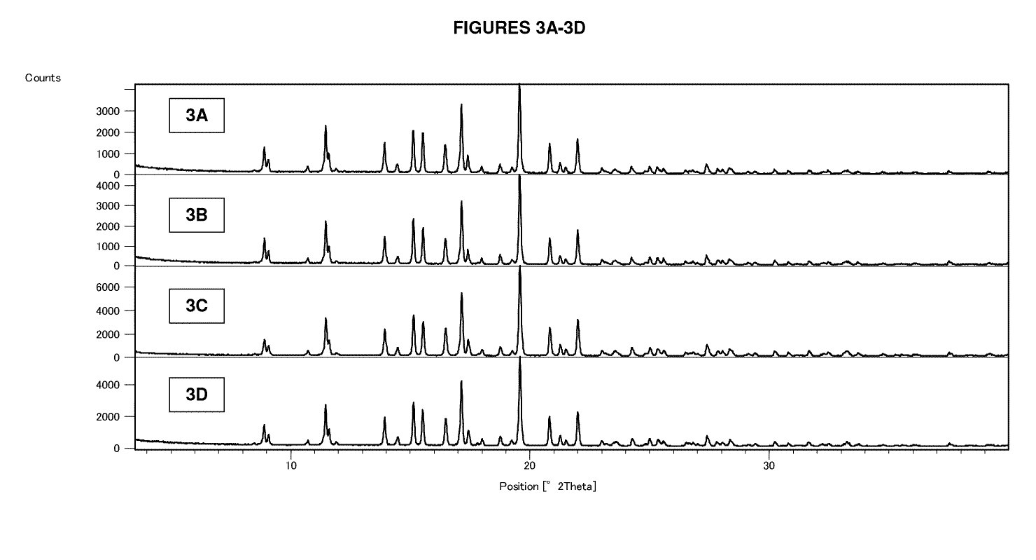

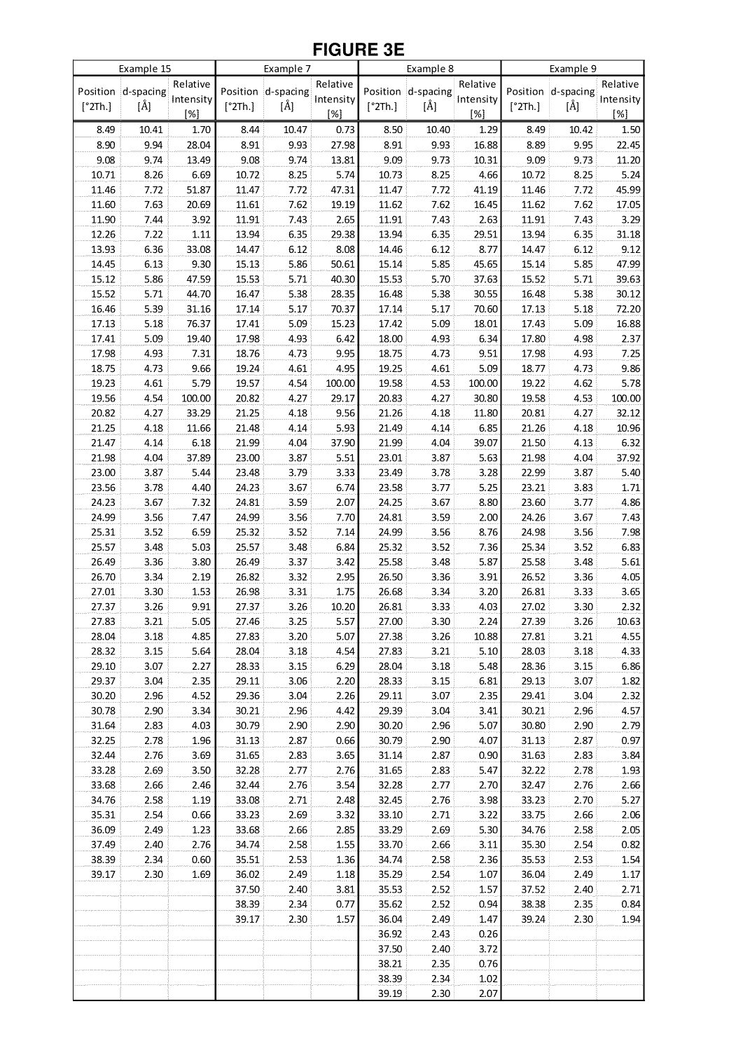

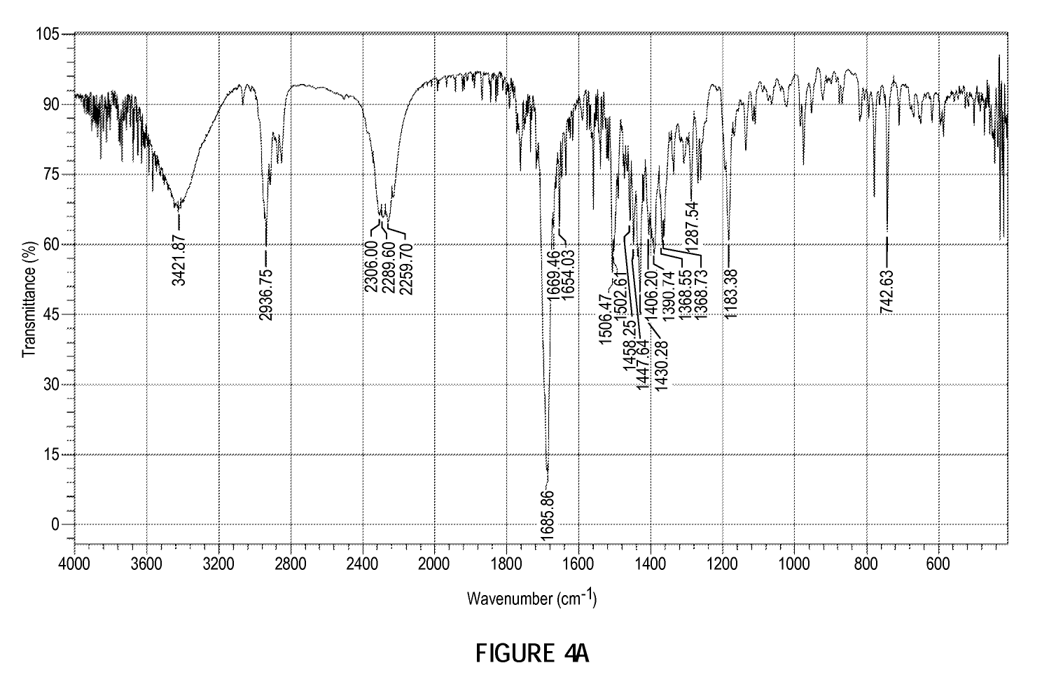

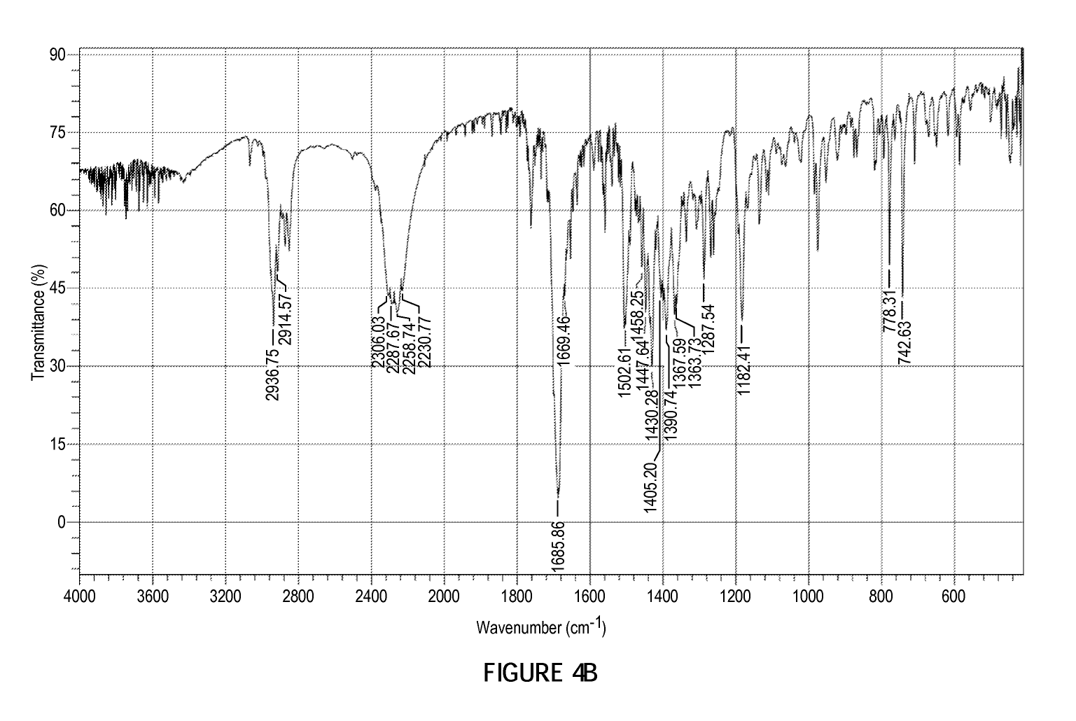

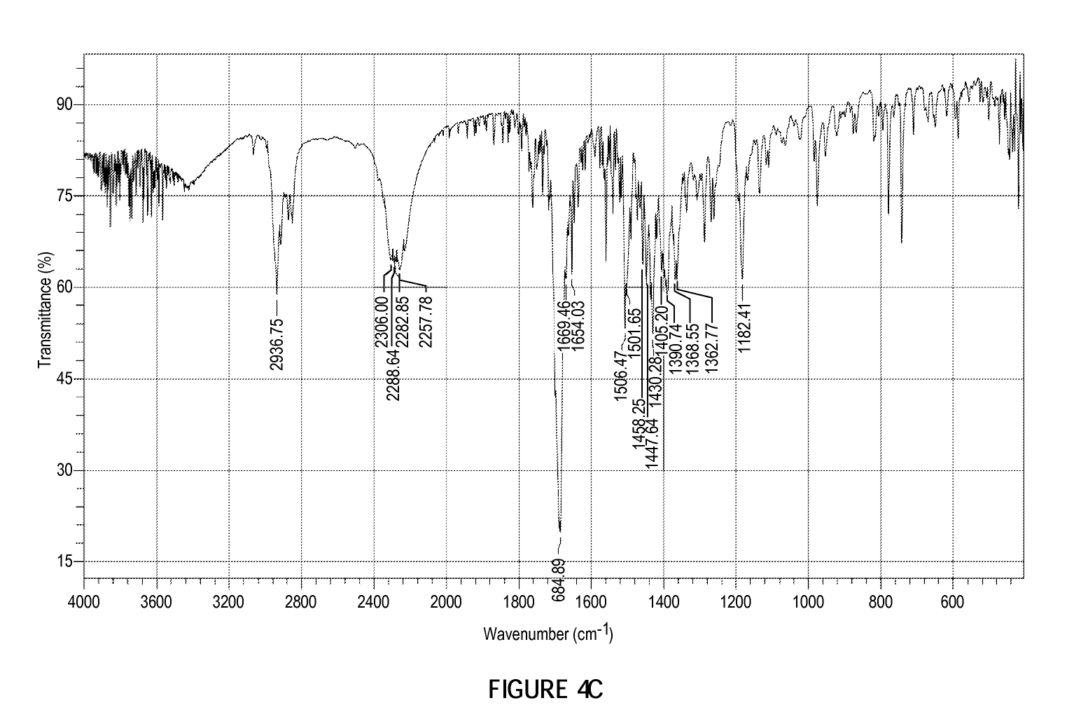

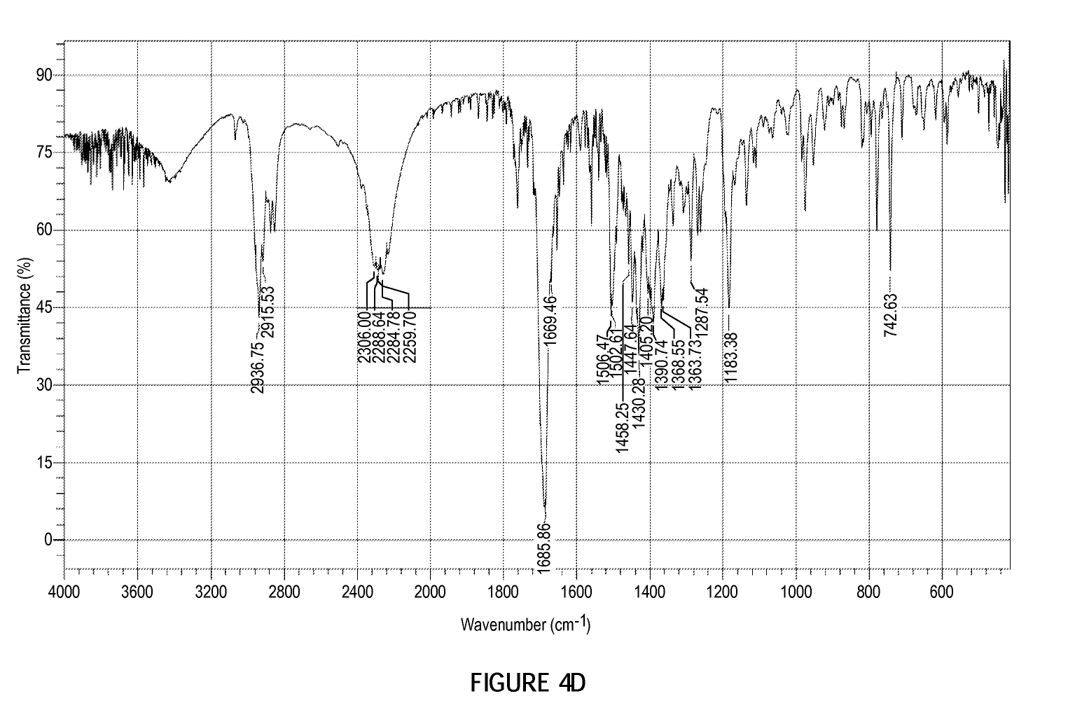

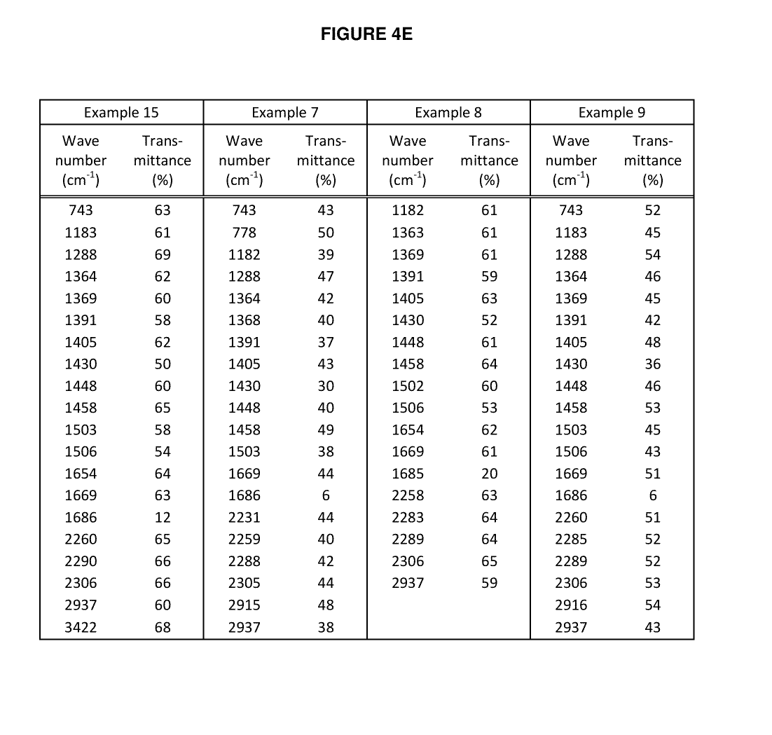

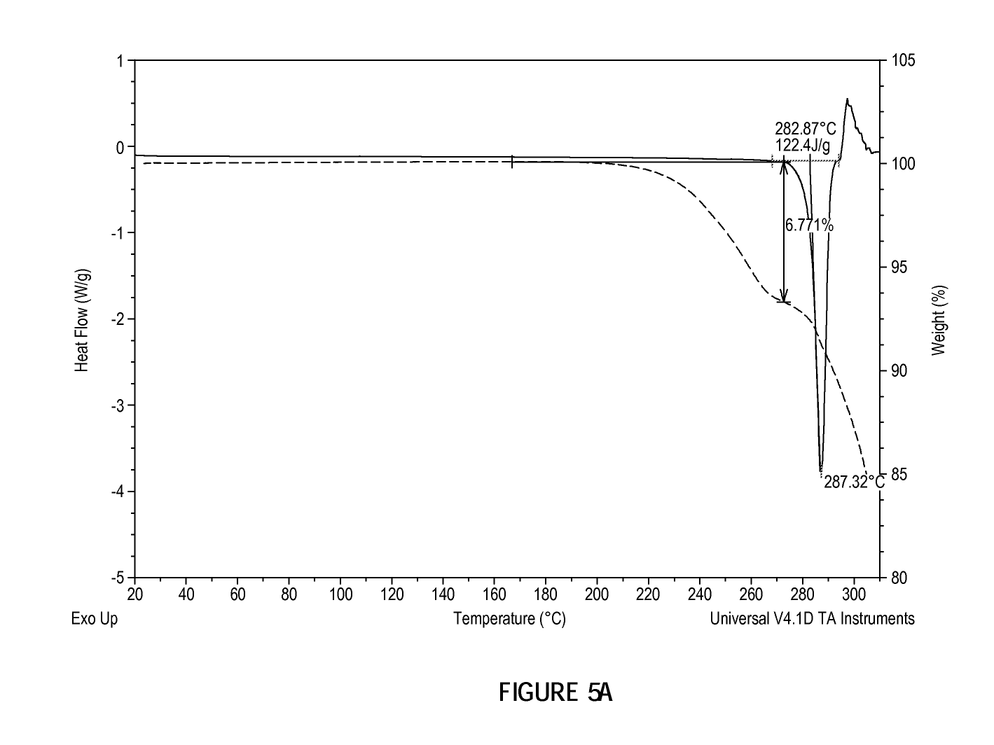

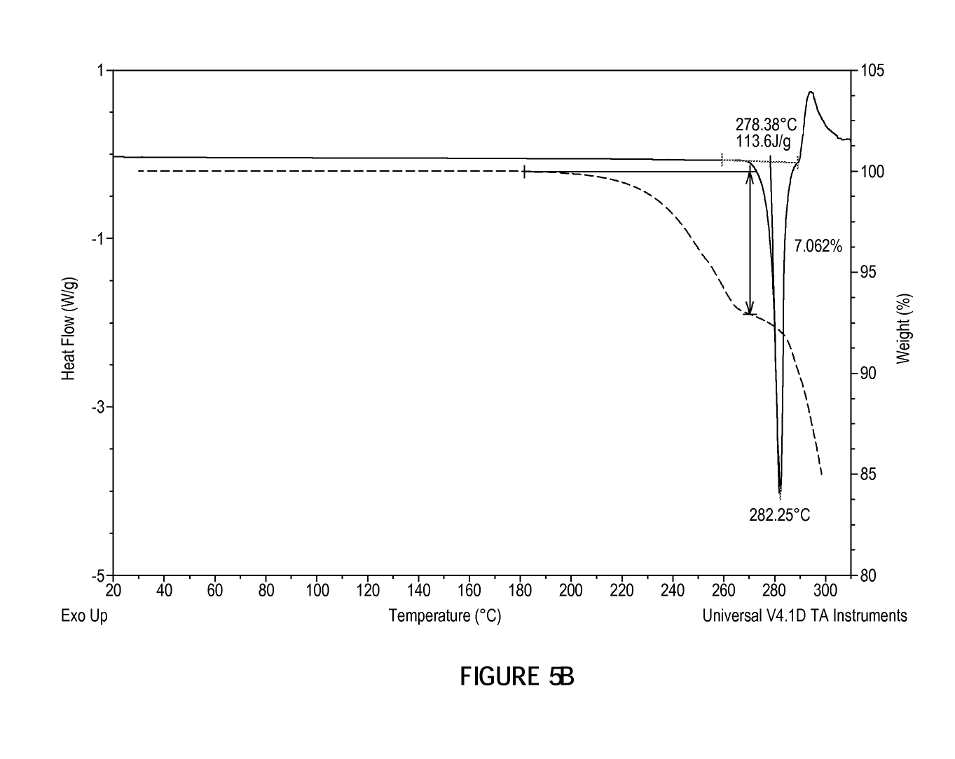

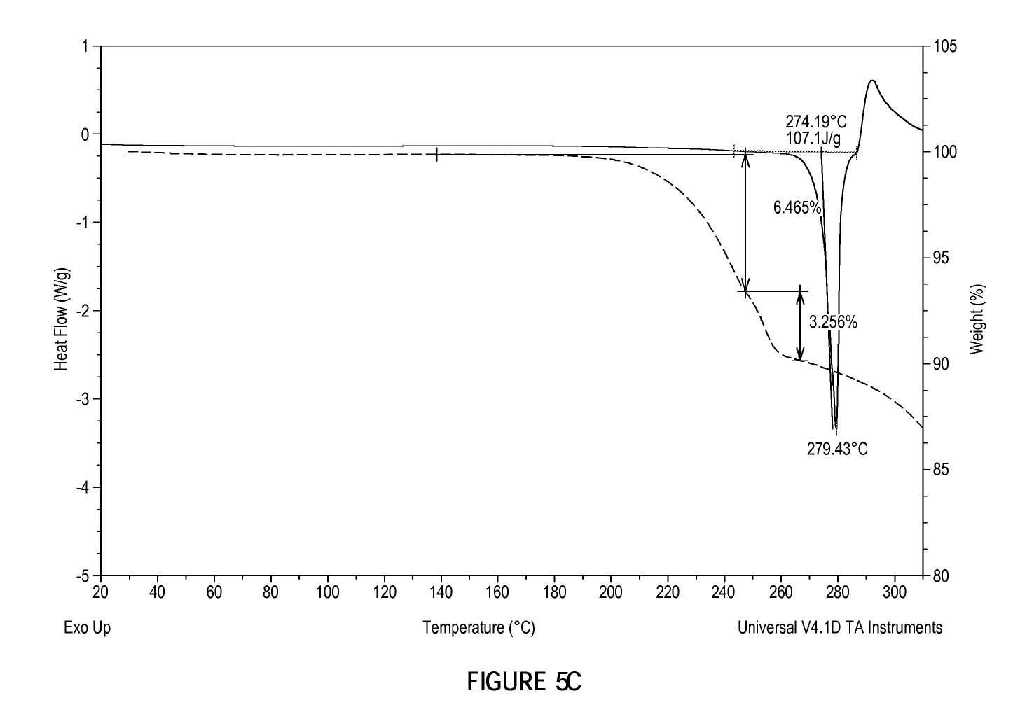

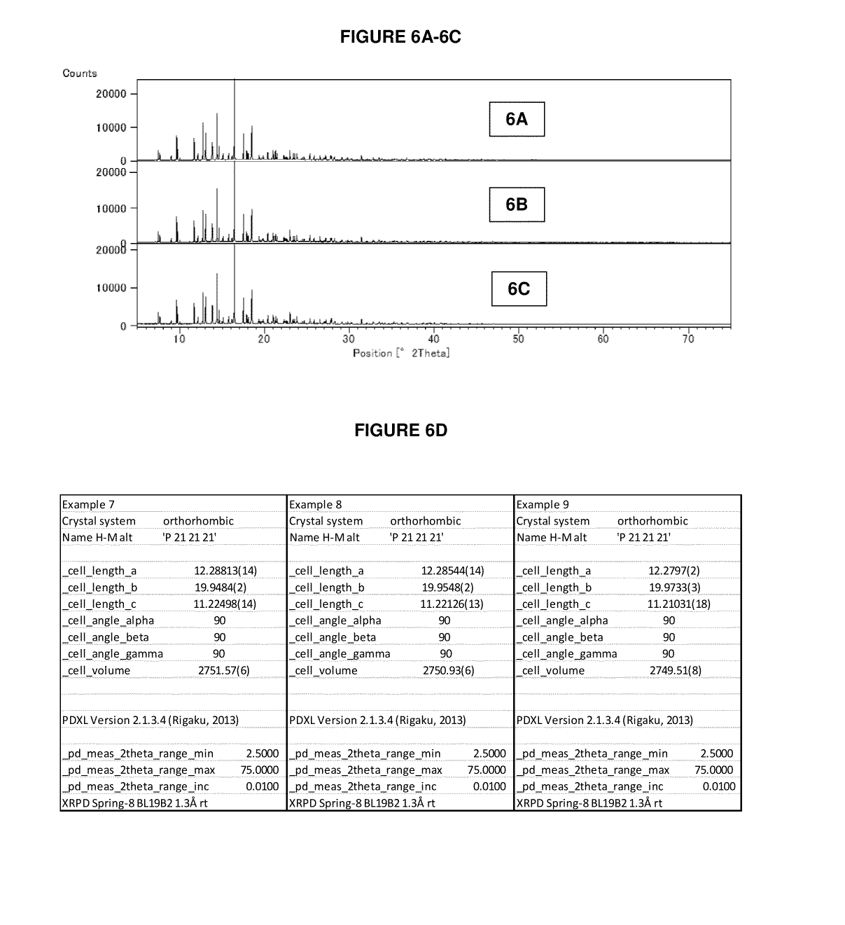

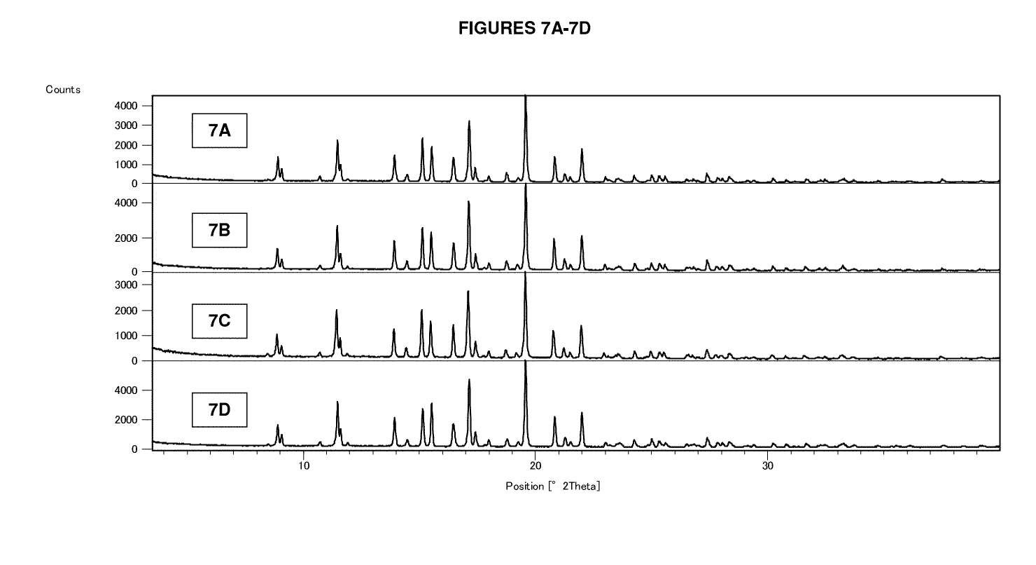

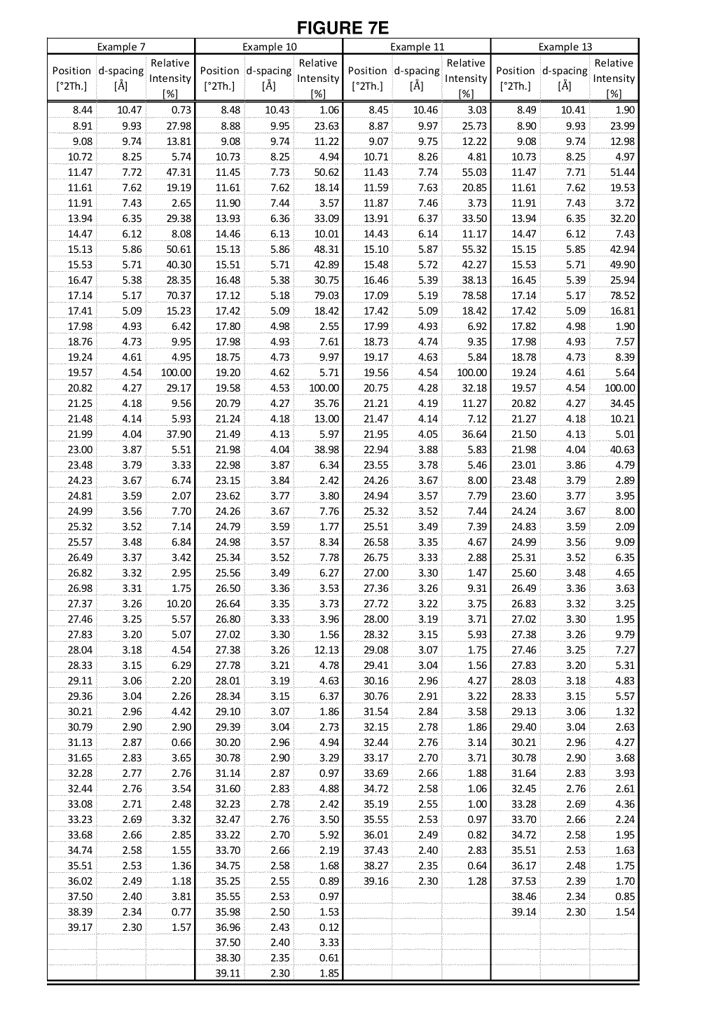

The XRPD pattern for the solid lurasidone HCl of Example 7 is shown in FIG. 3B and the peak information)(2θ(°), d-spacing, relative intensity) is provided in FIG. 3E. Also, the same pattern and data is repeated in FIGS. 7A and 7E. The IR spectrum is shown in FIG. 4B and the peak information (wavenumber, percent transmittance) is provided in FIG. 4E. The differential scanning calorimetry (DSC) thermogram and thermogravimetric analysis (TGA) graph are shown in FIG. 5A. An XRPD pattern collected at SPring-8 using synchrotron radiation is shown in FIG. 6A and the lattice parameters are shown in FIG. 6D.

### Example 8

Lurasidone (2.0 g) was dissolved in 2-propanol (200 g) with heating at about 80° C. to give a 2-propanol solution. To this solution was added a 14.4% hydrochloric acid (1.54 g) at about 80° C., and the reaction mixture was cooled to 0° C. The reaction mixture was filtered, and the resulting solid was dried under reduced pressure at room temperature to give lurasidone hydrochloride (1.97 g, yield: 92%). HPLC analysis of the product demonstrated the purity was 99.92%.

The XRPD pattern for the solid lurasidone HCl of Example 8 is shown in FIG. 3C and the peak information)(2θ(°), d-spacing, relative intensity) is provided in FIG. 3E. The IR spectrum is shown in FIG. 4C and the peak information (wavenumber, percent transmittance) is provided in FIG. 4E. The differential scanning calorimetry (DSC) thermogram and thermogravimetric analysis (TGA) graph are shown in FIG. 5B. An XRPD pattern collected at SPring-8 using synchrotron radiation is shown in FIG. 6B and the lattice parameters are shown in FIG. 6D.

### Example 9

Lurasidone (10 g) was added to 2-propanol (100 mL) at ambient temperature. The reaction mixture was cooled to about 0° C. to about 10° C. 7% Aqueous hydrochloric acid solution (11.6 g) was slowly added. The reaction mixture was stirred at about 5° C. to about 15° C. for about 4 hours and 30 minutes. The solid thus obtained, was filtered, washed with 2-propanol (2×10 mL) and dried at about 45° C. under reduced pressure for about 17 hours (10.7 g, yield: 99.7%). HPLC analysis of the product demonstrated the purity was 99.85%.

The XRPD pattern for the solid lurasidone HCl of Example 9 is shown in FIG. 3D and the peak information)(2θ(°), d-spacing, relative intensity) is provided in FIG. 3E. The IR spectrum is shown in FIG. 4D and the peak information (wavenumber, percent transmittance) is provided in FIG. 4E. The differential scanning calorimetry (DSC) thermogram and thermogravimetric analysis (TGA) graph are shown in FIG. 5C. An XRPD pattern collected at SPring-8 using synchrotron radiation is shown in FIG. 6C and the lattice parameters are shown in FIG. 6D.

### Example 10

A reaction mixture containing 4.0 g of pure lurasidone base and dry hydrogen chloride in 2-propanol (0.5 mol/L, 20 mL) was heated to a temperature of about 40° C. The reaction mixture was cooled to ambient temperature, diluted with 2-propanol (40 mL) and further stirred for about 5 hours. The solid material was filtered, washed with 2-propanol (15 mL) and dried under reduced pressure at about 45° C. to obtain a crystalline form of lurasidone hydrochloride (“Form 1”) as a white solid (4.53 g, yield: 105%).

The XRPD pattern for the solid lurasidone HCl of Example 10 (“Form 1”) is shown in FIG. 7B and the peak information)(2θ(°), d-spacing, relative intensity) is provided in FIG. 7E.

### Example 11

4.0 g of lurasidone was partly dissolved in ethyl acetate (40 mL) by heating at a temperature of about 40° C. The mixture was cooled to about 0° C. to 5° C. Dry hydrogen chloride in 2-propanol (0.5 mol/L, 20 mL) was added dropwise. The temperature was raised to ambient temperature and the contents were stirred for about 3.5 hours. The solid material was filtered, washed with ethyl acetate (10 mL) and dried under reduced pressure at about 45° C. to obtain a crystalline form of lurasidone hydrochloride (“Form 2”) as a white solid (4.53 g, yield: 105%).

The XRPD pattern for the solid lurasidone HCl of Example 11 (“Form 2”) is shown in FIG. 7C and the peak information)(2θ(°), d-spacing, relative intensity) is provided in FIG. 7E.

### Example 12

### Preparation of Crude Lurasidone Base

A reaction mixture containing 4-(1,2-Benzisothiazol-3-yl)piperazinium-1-spiro-(3′R,4′R)-3′,4′-tetramethylene-1′-pyrrolidine methanesulfonate (Compound (C)) (3.0 g), bicyclo[2.2.1]heptane-2-exo-3-exo-dicarboximide (Compound (D)) (1.7 g), dibenzo-18-crown-6 (0.03 g) and potassium carbonate (1.4 g) in xylene (40 mL) was refluxed for about 24 hours. The contents were filtered at about 50° C. and concentrated under reduced pressure at a temperature of about 70° C. to obtain crude lurasidone base as a solid (3.49 g).

Preparation of Lurasidone Hydrochloride from Crude Lurasidone Base:

Crude lurasidone base (3.0 g) was dissolved in acetone (30 mL) by heating the reaction mixture at a temperature of about 55° C. followed by dropwise addition of 4% aqueous hydrochloric acid solution (6 mL). The reaction mixture was stirred for about 1 hour and then cooled to about 0° C. Solvent was recovered completely from the reaction mixture. Fresh acetone (50 mL) was added and the reaction mixture was dried over anhydrous sodium sulphate (1.0 g). Diisopropyl ether (5 mL) was added dropwise to the reaction mixture. Turbidity was not observed. The reaction mixture was cooled to about 0° C. to −5° C. Turbidity was observed but no crystalline solid was obtained.

### Example 13

1.0 g of lurasidone was partly dissolved in ethyl acetate (10 mL) by heating the reaction mixture at a temperature of about 40° C. followed by dropwise addition of about 7% aqueous hydrogen chloride solution (5 mL). The reaction mixture was cooled to ambient temperature, diluted with ethyl acetate (15 mL) and stirred for about 2 hours. The solid material was filtered, washed with ethyl acetate (10 mL) and dried under reduced pressure at about 45° C. to obtain a crystalline form of lurasidone hydrochloride (“Form 4”) as a white solid (1.02 g, yield: 95.3%).

The XRPD pattern for the solid lurasidone HCl of Example 13 (“Form 4”) is shown in FIG. 7D and the peak information)(2θ(°), d-spacing, relative intensity) is provided in FIG. 7E.

### Example 14

Lurasidone (20.0 g) was dissolved in ethyl acetate (360 g) and water (2 g) with heating at about 55° C. to give an ethyl acetate solution. To this solution was added a 36% hydrochloric acid (9.04 g) at about 55° C. The reaction mixture was cooled to about 20° C. and stirred at the same temperature for about 40 minutes. The reaction mixture was filtered, and the resulting solid was washed with ethyl acetate (2×26 g) and dried under reduced pressure at room temperature to give lurasidone dihydrochloride salt (23.8 g, yield: 99%).

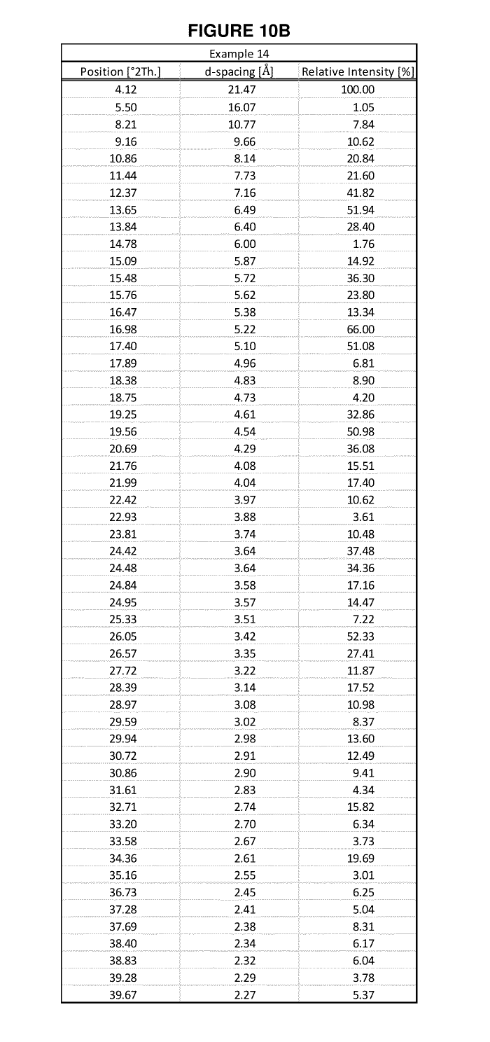

The XRPD pattern for the solid lurasidone dihydrochloride of Example 14 is shown in FIG. 10A, and the peak information)(2θ(°), d-spacing, relative intensity) is provided in FIG. 10B. The XRPD data for a sample that was powdered in an agate mortar and mounted on a glass plate was collected using an Ultima III (Rigaku) with a Cu source (1.54 Å) at a voltage of 40 kV and a current of 50 mA.

### Example 15

Lurasidone (61.0 kg) was dissolved in acetone (671 kg) with heating under reflux to give an acetone solution thereof. This solution was added dropwise to a 3.66% aqueous hydrochloric acid solution (137 kg, 1.1 equivalents) over a period of 35 min while the solution was kept at about 55° C. After the addition was completed, acetone (79.3 kg) and seed crystals (20 g) were added and the reaction mixture was stirred at about 60° C. for one hour. As seed crystals, lurasidone hydrochloride prepared by Example 7 can be used. Lurasidone hydrochloride as prepared by this Example 15 can also be used as seed crystals in subsequent preparations. The reaction mixture was cooled to 3° C., and stirred at the same temperature for 1.5 hr. The mixture was filtered and washed with acetone (2×100 L), and the resulting solid was dried by passing nitrogen gas (25° C.) through the pressure filter to give lurasidone hydrochloride (55.5 kg, yield: 84.7%). HPLC analysis of the product demonstrated the purity was 99.95%.

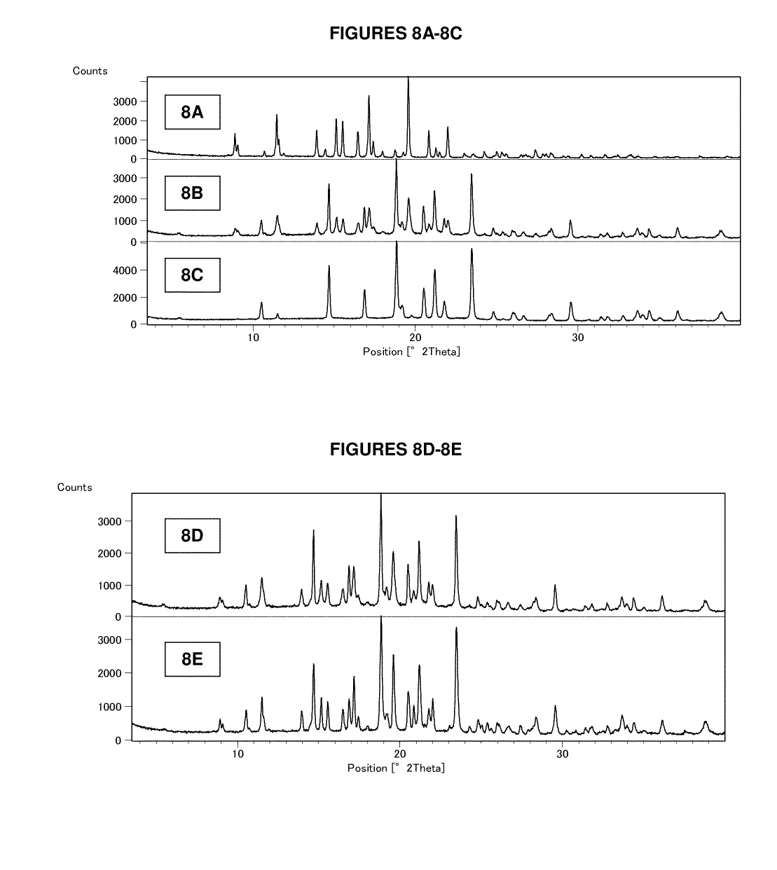

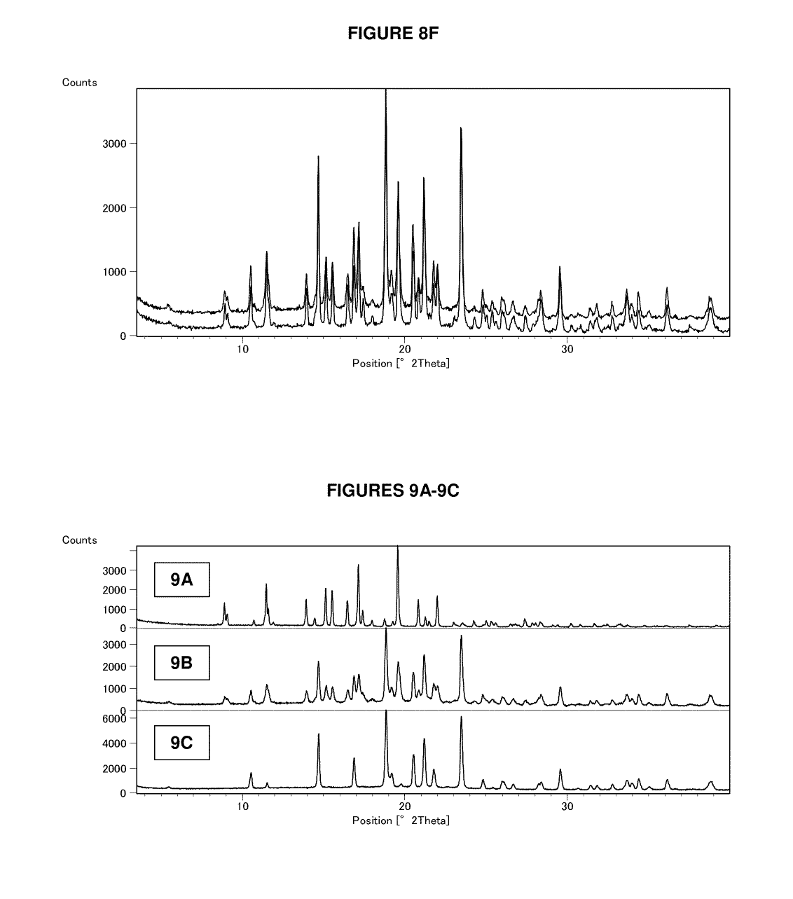

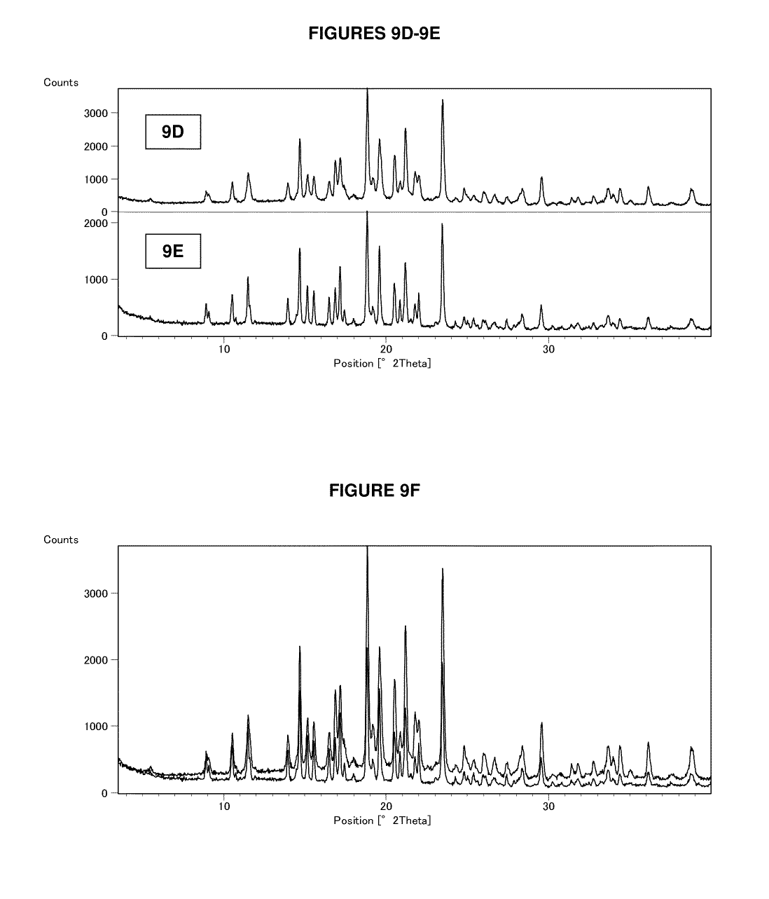

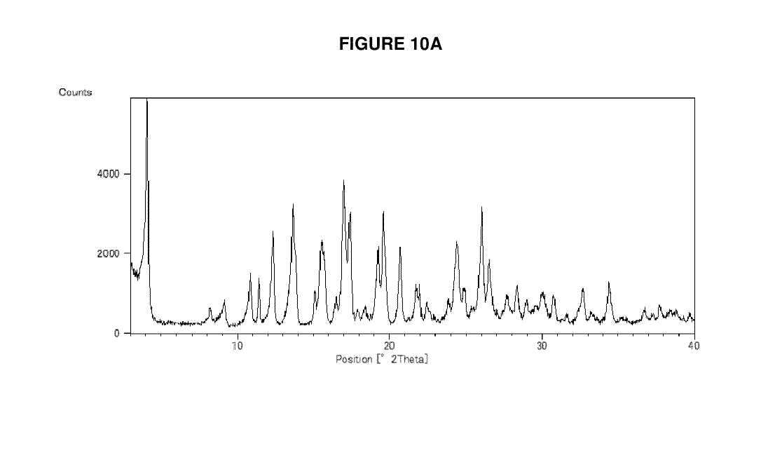

The XRPD pattern for the solid lurasidone HCl of Example 15 is shown in FIG. 3A, and the peak information)(2θ(°), d-spacing, relative intensity) is provided in FIG. 3E. The IR spectrum is shown in FIG. 4A and the peak information (wavenumber, percent transmittance) is provided in FIG. 4E. The XRPD pattern for the solid lurasidone HCl of Example 15 is also shown in FIGS. 8A and 9A.

### X-Ray Powder Diffraction Studies

Crystalline lurasidone hydrochloride was prepared by several methods as described above and examined by XRPD to determine whether different forms or polymorphs could be prepared. FIGS. 3A-3E compare the patterns for the lurasidone hydrochloride samples prepared according to Examples 15, 7, 8, and 9. The methods used to prepare lurasidone hydrochloride correspond as follows: Example 15 was the method used by Dainippon Sumitomo Pharma for the production of LATUDA®, when it was launched for sale in the United States as of Feb. 4, 2011; Example 7 replicates Example 14 of PCT Publication WO 2005/009999 (Kakiya et al.; corresponding U.S. national stage pre-grant publication is US 2006/0194970 A1); Example 8 replicates Example 17 of PCT Publication WO 2005/009999 (Kakiya et al.); and Example 9 replicates the example provided in PCT Publication WO 2013/030722 (Jayachandra et al.). The diffraction patterns in FIGS. 3A-3D and the data in the table of FIG. 3E reveal that the crystalline forms of lurasidone HCl produced according to Examples 15, 7, 8, and 9 are not distinguishable from one another.

The similarity of the crystalline forms of these preparations is further demonstrated by the IR spectra of solid samples of lurasidone hydrochloride salts prepared by Examples 15, 7, 8, and 9, shown in FIGS. 4A-4E, and in the DSC and TGA analyses of Examples 7, 8, and 9, shown in FIGS. 5A-5C. Given the lack of any significant differences among these measurements and the essentially identical diffraction patterns, the crystalline forms of lurasidone HCl salts prepared according to Examples 15, 7, 8, and 9 are the same.

FIGS. 6A-6D compare the XRPD patterns and cell parameters for Examples 7, 8, 9. The data in these figures was collected using synchrotron radiation as the X-ray source (wavelength: 1.299 Å). Here too, the XRPD patterns are essentially identical. The crystalline forms have the same crystal system (orthorhombic), the same space group (P 21 21 21), and essentially the same unit cell dimensions and volume. Comparing these lattice parameters, it is evident that the three production methods of Examples 7, 8, and 9 yield the same crystalline form.

FIGS. 7A-7E compare the patterns for the lurasidone hydrochloride samples prepared according to Examples 7, 10, 11, and 13. The methods used to prepare lurasidone hydrochloride correspond as follows: Example 7 replicates Example 14 of PCT Publication WO 2005/009999 (Kakiya et al.); Example 10 replicates Example 3 of PCT Publication WO 2012/107890 (Jayachandra et al.)(“Form 1”); Example 11 replicates Example 6 of PCT Publication WO 2012/107890 (Jayachandra et al.)(“Form 2”); and Example 13 replicates Example 9 of PCT Publication WO 2012/107890 (Jayachandra et al.)(“Form 4”). The diffraction patterns in FIGS. 7A-7D and the data in the table of FIG. 7E reveal that the crystalline forms of lurasidone HCl produced according to Examples 7, 10, 11, and 13 are not distinguishable from one another. In addition, Example 12, which replicated Example 8 of PCT Publication WO 2012/107890 (Jayachandra et al.), yielded no crystalline solid form (“Form 3”) of lurasidone HCl.

In another study, the X-ray powder diffraction pattern of lurasidone hydrochloride prepared according to Example 15 is compared with that of lurasidone hydrochloride found in commercially-available tablet form. The study was performed using the two commercially available products of LATUDA®, the 40 mg tablet and the 80 mg tablet. In the study, a milled tablet of the commercially available product was compared with a placebo tablet (prepared as described above, where mannitol replaces lurasidone hydrochloride) that was similarly milled. In addition, a sample of the placebo combined with the lurasidone hydrochloride of Example 15 was examined to test whether the juxtaposed diffraction patterns match that of the commercial product. To prepare samples of the latter type, 10 mg of lurasidone hydrochloride from Example 15 was powdered in an agate mortar and triturated with 30 mg of milled placebo tablet.

FIGS. 8A-8C show the analysis for the 40 mg tablet product, where the compound of Example 15 (same pattern as shown in FIG. 3A) is shown in FIG. 8A, the milled 40 mg tablet of LATUDA® (Lot No. G08313) is shown in FIG. 8B, and the milled placebo tablet is shown in FIG. 8C. To determine whether the XRPD pattern for the commercially available product (8B) is a combination of the lurasidone hydrochloride and the placebo tablet, a sample was formed by mixing lurasidone hydrochloride of Example 15 with the placebo tablet in a 1:3 ratio. The patterns for the milled commercial tablet and the mixture of compound with placebo tablet are shown in FIGS. 8D and 8E, respectively. The two patterns are virtually identical, indicating that the lurasidone hydrochloride present in the commercially available tablet and that prepared according to Example 15 have the same crystalline form. These two spectra are overlaid in FIG. 8F, wherein the correspondence of the peaks in these two spectra is apparent.

The same study was performed for the 80 mg tablet. FIGS. 9A-9C show the analysis for the 80 mg tablet product, where the compound of Example 15 (same pattern as shown in FIG. 3A) is shown in FIG. 9A, the milled 80 mg tablet of LATUDA® (Lot No. G08264) is shown in FIG. 9B, and the milled placebo tablet is shown in FIG. 9C. To determine whether the XRPD pattern for the commercially available product (9B) is a combination of the lurasidone hydrochloride and the placebo tablet, a sample was formed by mixing lurasidone hydrochloride of Example 15 with the placebo tablet in a 1:3 ratio. The patterns for the milled commercial tablet and the mixture of compound with placebo tablet are shown in FIGS. 9D and 9E, respectively. The two patterns are virtually identical, indicating that the lurasidone hydrochloride present in the commercially available tablet and that prepared according to Example 15 have the same crystalline form. These two spectra are overlaid in FIG. 9F, wherein the correspondence of the peaks in these two spectra is apparent.

### INDUSTRIAL APPLICABILITY

The process of the present invention is a process for preparing quaternary ammonium salt (4) in steady reaction time and in steady quality, thus it has some merits, in particular for the industrial purpose.

1. A process for preparing a quaternary ammonium salt of formula (4): 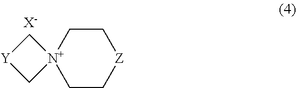
wherein
X is a halogen atom, C₁₋₆ alkylsulfonyloxy group, or C₆₋₁₀ arylsulfonyloxy group, and X⁻ is a counteranion thereof,
Y is a substituent of the following formula (3a) or (3b): 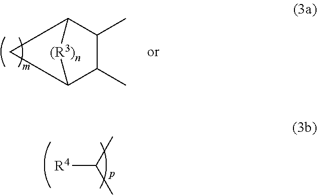
wherein R³ is independently methylene or oxygen atom; R⁴ is independently C₁₋₆ alkyl group, C₁₋₆ alkoxy group, or hydroxy group; m and n are independently 0, 1, 2, or 3; and p is 1 or 2, and
Z is ═N—R¹ or ═CH—R² wherein R¹ is C₁₋₆ alkyl group, C₃₋₇ cycloalkyl group, C₅₋₇ cycloalkenyl group, C₆₋₁₀ aryl group, or 5- to 10-membered monocyclic or bicyclic heteroaryl group; R² is C₁₋₆ alkyl group, C₁₋₆ alkoxy group, C₁₋₆ alkylthio group, C₃₋₇ cycloalkyl group, C₃₋₇ cycloalkyloxy group, C₃₋₇ cycloalkylthio group, C₅₋₇ cycloalkenyl group, C₅₋₇ cycloalkenyloxy group, C₅₋₇ cycloalkenylthio group, C₆₋₁₀ aryl group, C₆₋₁₀ aryloxy group, C₆₋₁₀ arylthio group, 5- to 10-membered monocyclic or bicyclic heteroaryl group, 5- to 10-membered monocyclic or bicyclic heteroaryloxy group, or 5- to 10-membered monocyclic or bicyclic heteroarylthio group,
comprising reacting a compound of formula (1): 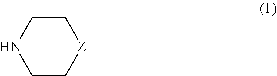
wherein Z is as defined above
with 1 to 2 mole of a compound of formula (2): 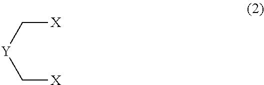
wherein X is independently selected from the above-defined ones, and Y is as defined above, per one mole of the compound of formula (1)
in the presence of 1 to 5 mole of a phosphate per one mole of the compound of formula (1) and 0.01 to 0.1 part by weight of water per one part by weight of the phosphate.

2. The process of claim 1 wherein X is independently C₁₋₆ alkylsulfonyloxy group, or C₆₋₁₀ arylsulfonyloxy group.

3. The process of claim 2 wherein X is methanesulfonyloxy group.

4. The process of claim 1 wherein Y is the substituent of formula (3a).

5. The process of claim 4 wherein m is 2 and n is 0.

6. The process of claim 1 wherein Z is ═N—R¹.

7. The process of claim 6 wherein R¹ is 5- to 10-membered monocyclic or bicyclic heteroaryl group.

8. The process of claim 7 wherein R¹ is 1,2-benzisothiazol-3-yl.

9. The process of claim 1 wherein the phosphate is dibasic potassium phosphate.

10. The process of claim 1 wherein the phosphate is 1 to 3 mole per one mole of the compound of formula (1).

11. The process of claim 1 wherein the amount of water is 0.01 to 0.05 part by weight per one part by weight of the phosphate.

12. The process of claim 1 wherein the compound of formula (1) is 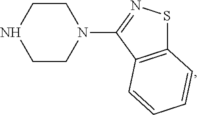 [SMILES: c1ccc2c(N3CCNCC3)nsc2c1]
the compound of formula (2) is 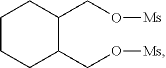 [SMILES: CS(=O)(=O)OCC1CCCCC1COS(C)(=O)=O]
and
the quaternary ammonium salt of formula (4) is

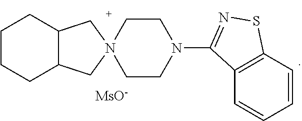

13. A process for preparing a compound of formula (8): 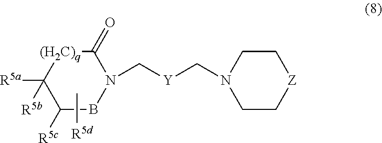
wherein
B is carbonyl group or sulfonyl group,
R^{5a}, R^{5b}, R^{5c}, and R^{5d} are independently hydrogen atom or C₁₋₄ alkyl group, alternatively R^{5a} and R^{5b}, or R^{5a} and R^{5c} may be taken together to form a hydrocarbon ring, or R^{5a} and R^{5c} may be taken together to form an aromatic hydrocarbon ring, wherein the hydrocarbon ring may be bridged with C₁₋₄ alkylene or oxygen atom wherein the C₁₋₄ alkylene and the hydrocarbon ring may be substituted with at least one C₁₋₄ alkyl,
q is 0 or 1, and
Y and Z are as defined in Term 1,
comprising reacting the quaternary ammonium salt (4) prepared according to any one of claims 1 to 12 with the following compound (7): 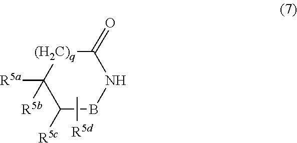
wherein B, R^{5a}, R^{5b}, R^{5c}, R^{5d}, and q are as defined above, in the presence of a solid inorganic base.

14. The process of claim 13 wherein B is carbonyl group.

15. The process of claim 13 wherein R^{5a} and R^{5c} are taken together to form a hydrocarbon ring which may be bridged with C₁₋₄ alkylene, and R^{5b} and R^{5d} are hydrogen atom.

16. The process of claim 15 wherein Compound (7) is the following compound of formula (7b):

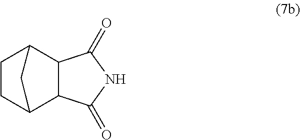 [SMILES: O=C1NC(=O)C2C3CCC(C3)C12]

17. The process of claim 13 wherein Compound (8) is (3aR,4S,7R,7aS)-2-{(1R,2R)-2-[4-(1,2-benzisothiazol-3-yl) piperazin-1-ylmethyl]cyclohexyl-methyl}hexahydro-4,7-methano-2H-isoindole-1,3-dione.
